(19) United States
(12) Patent Application Publication
(10) Pub. No.: US 2024/0026001 A1
(43) Pub. Date: Jan. 25, 2024

(54) USE OF ANTI-IL-27 ANTIBODIES

(71) Applicants: SURFACE ONCOLOGY, INC., Cambridge, MA (US); MSD INTERNATIONAL GMBH, Luzern 6 (CH)

(72) Inventors: Alison O'NEILL, Cambridge, MA (US); Lauren HARSHMAN, Cambridge, MA (US); Jonathan HILL, Cambridge, MA (US); Jou-Ku CHUNG, Cambridge (US); Kerry WHITE, Cambridge, MA (US); Robert ROSS, Cambridge, MA (US)

(21) Appl. No.: 18/201,620

(22) Filed: May 24, 2023

Related U.S. Application Data
(60) Provisional application No. 63/345,758, filed on May 25, 2022.

(51) Int. Cl. C07K 16/28; C07K 16/24; A61P 35/00

(52) U.S. Cl. CPC C07K 16/2818; C07K 16/244; A61P 35/00; C07K 2317/24; A61K 2039/507

(57) ABSTRACT

The present disclosure relates to methods of treating cancer comprising administering to a subject an anti-IL-27 antibody and pembrolizumab.

### RELATED APPLICATIONS

This application claims priority to U.S. Provisional Patent Application Ser. No. 63/345,758, filed May 25, 2022, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on Sep. 19, 2023, is named 743232_SRF9-002_ST26.xml and is 44,170 bytes in size.

### FIELD

The present disclosure relates generally to methods for treating a subject afflicted with a cancer by administering to the subject an anti-IL-27 antibody and pembrolizumab (an anti-PD-1 antibody).

### BACKGROUND

In recent years, an increasing body of evidence suggests that the immune system operates as a significant barrier to tumor formation and progression. The principle that naturally occurring T cells with anti-tumor potential or activity exist in a patient with cancer has rationalized the development of immunotherapeutic approaches in oncology. Immune cells, such as T cells, macrophages, and natural killer cells, can exhibit anti-tumor activity and effectively control the occurrence and growth of malignant tumors. Tumor-specific or -associated antigens can induce immune cells to recognize and eliminate malignancies (Chen & Mellman, (2013) Immunity 39(1):1-10). In spite of the existence of tumor-specific immune responses, malignant tumors often evade or avoid immune attack through a variety of immunomodulatory mechanisms resulting in the failure to control tumor occurrence and progression (Motz & Coukos, (2013) Immunity 39(1):61-730). Indeed, an emerging hallmark of cancer is the exploitation of these immunomodulatory mechanisms and the disablement of anti-tumor immune responses, resulting in tumor evasion and escape from immunological killing (Hanahan and Weinberg (2011) Cell 144(5):646-674).

Despite the significant advances being made in cancer treatment and management, there is still an ongoing need for new and effective therapies for treating and managing cancer.

### SUMMARY OF THE DISCLOSURE

Some aspects of the present disclosure are directed to a method of stimulating an immune response in a subject, the method comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Some aspects of the present disclosure are directed to a method of treating a cancer in a subject in need thereof comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Some aspects of the present disclosure are directed to an antibody that binds human IL-27 or an antigen binding portion thereof (“an anti-IL-27 antibody”) for use in stimulating an immune response in a subject, wherein the anti-IL-27 antibody is co-administered with pembrolizumab; and wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Some aspects of the present disclosure are directed to pembrolizumab for use in stimulating an immune response in a subject, wherein pembrolizumab is co-administered with an antibody that binds human IL-27 or an antigen binding portion thereof (“an anti-IL-27 antibody”); and wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Some aspects of the present disclosure are directed to an antibody that binds human IL-27 or an antigen binding portion thereof (“an anti-IL-27 antibody”) and pembrolizumab for use in stimulating an immune response in a subject; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Some aspects of the present disclosure are directed to an antibody that binds human IL-27 or an antigen binding portion thereof (“an anti-IL-27 antibody”) for use in treating cancer in a subject, wherein the anti-IL-27 antibody is co-administered with pembrolizumab; and wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Some aspects of the present disclosure are directed to pembrolizumab for use in treating cancer in a subject, wherein pembrolizumab is co-administered with an antibody that binds human IL-27 or an antigen binding portion thereof (“an anti-IL-27 antibody”); and wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Some aspects of the present disclosure are directed to an antibody that binds human IL-27 or an antigen binding portion thereof (“an anti-IL-27 antibody”) and pembrolizumab for use in treating cancer in a subject; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

In some aspects, the anti-IL-27 antibody is administered at a dose of at least about 0.003 mg/kg to at least about 20 mg/kg. In some aspects, the anti-IL-27 antibody is administered at a dose of at least about 0.003 mg/kg, at least about 0.006 mg/kg, at least about 0.009 mg/kg, at least about 0.03 mg/kg, at least about 0.06 mg/kg, at least about 0.09 mg/kg, at least about 0.3 mg/kg, at least about 0.6 mg/kg, at least about 0.9 mg/kg, at least about 1.0 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, or at least about 20 mg/kg.

In some aspects, the anti-IL-27 antibody is administered once about every week, once about every two weeks, once about every three weeks, once about every four weeks, once about every 6 weeks, once about every 8 weeks, or once about every 12 weeks. In some aspects, the anti-IL-27 antibody is administered once about every two weeks. In some aspects, the anti-IL-27 antibody is administered once about every three weeks. In some aspects, the anti-IL-27 antibody is administered once about every four weeks.

In some aspects, the anti-IL-27 antibody is administered at a dose of about 0.3 mg/kg once about every three weeks. In some aspects, the anti-IL-27 antibody is administered at a dose of about 1 mg/kg once about every three weeks. In some aspects, the anti-IL-27 antibody is administered at a dose of about 3 mg/kg once about every three weeks. In some aspects, the anti-IL-27 antibody is administered at a dose of about 6 mg/kg once about every three weeks. In some aspects, the anti-IL-27 antibody is administered at a dose of about 10 mg/kg once about every two weeks. In some aspects, the anti-IL-27 antibody is administered at a dose of about 10 mg/kg once about every three weeks. In some aspects, the anti-IL-27 antibody is administered at a dose of about 10 mg/kg once about every four weeks. In some aspects, the anti-IL-27 antibody is administered at a dose of about 13 mg/kg once about every three weeks. In some aspects, the anti-IL-27 antibody is administered at a dose of about 16 mg/kg once about every three weeks. In some aspects, anti-IL-27 antibody is administered at a dose of about 20 mg/kg once about every three weeks.

In some aspects, pembrolizumab is administered at a dose of at least about 1 mg/kg to at least about 10 mg/kg. In some aspects, pembrolizumab is administered at a dose of at least about 1 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, or at least about 10 mg/kg.

In some aspects, pembrolizumab is administered at a flat dose. In some aspects, pembrolizumab is administered at a flat dose of at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 210 mg, at least about 220 mg, at least about 230 mg, at least about 240 mg, at least about 250 mg, at least about 260 mg, at least about 270 mg, at least about 280 mg, at least about 290 mg, at least about 300 mg, at least about 310 mg, at least about 320 mg, at least about 340 mg, at least about 350 mg, at least about 360 mg, at least about 370 mg, at least about 380 mg, at least about 390 mg, at least about 400 mg, at least about 410 mg, at least about 420 mg, at least about 430 mg, at least about 440 mg, at least about 450 mg, at least about 460 mg, at least about 470 mg, at least about 480 mg, at least about 490 mg, at least about 500 mg, at least about 600 mg, at least about 700 mg, at least about 800 mg, at least about 900 mg, or at least about 1000 mg. In some aspects, pembrolizumab is administered once about every week, once about every two weeks, once about every three weeks, or once about every four weeks.

In some aspects, pembrolizumab is administered at a flat dose of at least about 200 mg once about every three weeks. In some aspects, pembrolizumab is administered at a flat dose of at least about 300 mg once about every six weeks. In some aspects, pembrolizumab is administered at a flat dose of at least about 400 mg once about every six weeks.

In some aspects, (i) the anti-IL-27 antibody is administered at a dose of about 0.3 mg/kg once about every three weeks; and (ii) pembrolizumab is administered at a flat dose of about 200 mg once about every three weeks. In some aspects, (i) the anti-IL-27 antibody is administered at a dose of about 1 mg/kg once about every three weeks; and (ii) pembrolizumab is administered at a flat dose of about 200 mg once about every three weeks. In some aspects, (i) the anti-IL-27 antibody is administered at a dose of about 3 mg/kg once about every three weeks; and (ii) pembrolizumab is administered at a flat dose of about 200 mg once about every three weeks. In some aspects, (i) the anti-IL-27 antibody is administered at a dose of about 6 mg/kg once about every three weeks; and (ii) pembrolizumab is administered at a flat dose of about 200 mg once about every three weeks. In some aspects, (i) the anti-IL-27 antibody is administered at a dose of about 10 mg/kg once about every three weeks; and (ii) pembrolizumab is administered at a flat dose of about 200 mg once about every three weeks. In some aspects, (i) the anti-IL-27 antibody is administered at a dose of about 13 mg/kg once about every three weeks; and (ii) pembrolizumab is administered at a flat dose of about 200 mg once about every three weeks. In some aspects, (i) the anti-IL-27 antibody is administered at a dose of about 16 mg/kg once about every three weeks; and (ii) pembrolizumab is administered at a flat dose of about 200 mg once about every three weeks. In some aspects, (i) the anti-IL-27 antibody is administered at a dose of about 20 mg/kg once about every three weeks; and (ii) pembrolizumab is administered at a flat dose of about 200 mg once about every three weeks.

In some aspects, the anti-IL-27 antibody and pembrolizumab are administered on the same day. In some aspects, the anti-IL-27 antibody and pembrolizumab are administered concurrently. In some aspects, the anti-IL-27 antibody and pembrolizumab are administered sequentially. In some aspects, the anti-IL-27 antibody is administered before pembrolizumab. In some aspects, the anti-IL-27 antibody is administered after pembrolizumab. In some aspects, the anti-IL-27 antibody and pembrolizumab are administered on different days.

In some aspects, the anti-IL-27 antibody comprises a heavy chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 11. In some aspects, the anti-IL-27 antibody comprises a heavy chain variable region comprising the amino acid sequence set forth in SEQ ID NO: 11. In some aspects, the anti-IL-27 antibody comprises a light chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 19. In some aspects, the anti-IL-27 antibody comprises a light chain variable region comprising the amino acid sequence set forth in SEQ ID NO: 19. In some aspects, the anti-IL-27 antibody comprises a heavy chain variable region comprising the amino acid sequence set forth in SEQ ID NO: 11 and a light chain variable region comprising the amino acid sequence set forth in SEQ ID NO: 19.

In some aspects, the anti-IL-27 antibody comprises a heavy chain comprising the amino acid sequence set forth in SEQ ID NO: 21. In some aspects, the anti-IL-27 antibody comprises a heavy chain comprising the amino acid sequence set forth in SEQ ID NO: 25. In some aspects, the anti-IL-27 antibody comprises a light chain comprising the amino acid sequence set forth in SEQ ID NO: 23. In some aspects, the anti-IL-27 antibody comprises a heavy chain comprising the amino acid sequence set forth in SEQ ID NO: 21 and a light chain comprising the amino acid sequence set forth in SEQ ID NO: 23. In some aspects, the anti-IL-27 antibody comprises a heavy chain comprising the amino acid sequence set forth in SEQ ID NO: 25 and a light chain comprising the amino acid sequence set forth in SEQ ID NO: 23.

In some aspects, the subject is afflicted with a cancer. In some aspects, the cancer is selected from non-small cell lung cancer), hepatocellular carcinoma (HCC), and renal cancer (e.g., renal cell carcinoma (RCC), e.g., clear cell RCC and/or non-clear cell RCC).

In some aspects, the cancer is refractory. In some aspects, the subject previously received at least one, at least two, at least three, at least four, or at least five prior anticancer therapies. In some aspects, the prior anticancer therapy comprises a prior anti-PD-1 antibody therapy. In some aspects, the cancer is advanced or metastatic.

### BRIEF DESCRIPTION OF THE DRAWINGS

FIG. 1: Graphical summary of patient tumor response to combination therapy comprising 10 mg/kg of IL-27 blocking antibody (SRF388) and 200 mg pembrolizumab every 3 weeks. Tumor response was assessed by RECIST1.1. Tumor response is presented as best percent change from baseline in sum of target lesions (n=9). The cancer type for each patient (ccRCC or HCC) is also depicted.

FIG. 2: Graphical summary of patient tumor response to combination therapy comprising 10 mg/kg of IL-27 blocking antibody (SRF388) and 200 mg pembrolizumab every 3 weeks. Tumor response was assessed by RECIST1.1. Tumor response is presented as target lesion change over time (n=9).

### DETAILED DESCRIPTION

Some aspects of the present disclosure are directed to methods of stimulating an immune response in a subject, the method comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15. Some aspects of the present disclosure are directed to methods of treating a cancer in a subject in need thereof comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15. In some aspects, the antibody or an antigen binding portion thereof that binds to human IL-27 is administered at a dose of at least about 0.003 mg/kg to at least about 20 mg/kg. In some aspects, pembrolizumab is administered at a dose of at least about 1 mg/kg to at least about 10 mg/kg. In some aspects, pembrolizumab is administered at a flat dose of at least about 200 mg once about every three weeks.

### I. Definitions

Terms used in the claims and specification are defined as set forth below unless otherwise specified.

It must be noted that, as used in the specification and the appended claims, the singular forms “a,” “an” and “the” include plural references unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The term “ameliorating” refers to any therapeutically beneficial result in the treatment of a disease state, e.g., cancer, including prophylaxis, lessening in the severity or progression, remission, or cure thereof.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, the term “amount” or “level” is used in the broadest sense and refers to a quantity, concentration or abundance of a substance (e.g., a metabolite, a small molecule, a protein, an mRNA, a marker). When referring to a metabolite or small molecule (e.g. a drug), the terms “amount”, “level” and “concentration” are generally used interchangeably and generally refer to a detectable amount in a biological sample. “Elevated levels” or “increased levels” refers to an increase in the quantity, concentration or abundance of a substance within a sample relative to a control sample, such as from an individual or individuals who are not suffering from the disease or disorder (e.g., cancer) or an internal control. In some aspects, the elevated level of a substance (e.g., a drug) in a sample refers to an increase in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g., HPLC). “Reduced levels” refers to a decrease in the quantity, concentration or abundance of a substance (e.g., a drug) in an individual relative to a control, such as from an individual or individuals who are not suffering from the disease or disorder (e.g., cancer) or an internal control. In some aspects, a reduced level is little or no detectable quantity, concentration or abundance. In some aspects, the reduced level of a substance (e.g., a drug) in a sample refers to a decrease in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, HPLC).

When referring to a protein, mRNA or a marker, such as those described herein, the terms “level of expression” or “expression level” in general are used interchangeably and generally refer to a detectable amount of a protein, mRNA, or marker in a biological sample. In some aspects, a detectable amount or detectable level of a protein, mRNA or a marker is associated with a likelihood of a response to an agent, such as those described herein. “Expression” generally refers to the process by which information contained within a gene is converted into the structures (e.g., a protein marker, such as PD-L1) present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). “Elevated expression,” “elevated expression levels,” or “elevated levels” refers to an increased expression or increased levels of a substance within a sample relative to a control sample, such as an individual or individuals who are not suffering from the disease or disorder (e.g., cancer) or an internal control. In some aspects, the elevated expression of a substance (e.g., a protein marker, such as PD-L1) in a sample refers to an increase in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g., FACS). “Reduced expression,” “reduced expression levels,” or “reduced levels” refers to a decrease expression or decreased levels of a substance (e.g., a protein marker) in an individual relative to a control, such as an individual or individuals who are not suffering from the disease or disorder (e.g., cancer) or an internal control. In some aspects, reduced expression is little or no expression. In some aspects, the reduced expression of a substance (e.g., a protein marker) in a sample refers to a decrease in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, FACS).

As used herein, the term “angiogenesis” or “neovascularization” refers to the process by which new blood vessels develop from pre-existing vessels (Varner et al., (1999) Angiogen. 3:53-60; Mousa et al., (2000) Angiogen. Stim. Inhib. 35:42-44; Kim et al., (2000) Amer. J. Path. 156:1345-1362 Kim et al., (2000) J. Biol. Chem. 275:33920-33928; Kumar et al. (2000) Angiogenesis: From Molecular to Integrative Pharm. 169-180). Endothelial cells from pre-existing blood vessels or from circulating endothelial stem cells (Takahashi et al., (1995) Nat. Med. 5:434-438; Isner et al., (1999) J. Clin. Invest. 103:1231-1236) become activated to migrate, proliferate, and differentiate into structures with lumens, forming new blood vessels, in response to growth factor or hormonal cues, or hypoxic or ischemic conditions. During ischemia, such as occurs in cancer, the need to increase oxygenation and delivery of nutrients apparently induces the secretion of angiogenic factors by the affected tissue; these factors stimulate new blood vessel formation. Several additional terms are related to angiogenesis.

The term “antagonist,” as used herein, refers to an inhibitor of a target molecule and may be used synonymously herein with the term “inhibitor.” As used herein, the term “antagonist” refers to any molecule that partially or fully blocks, inhibits, or neutralizes a biological activity of a native polypeptide disclosed herein. Suitable antagonist molecules specifically include antagonist antibodies or antibody fragments, fragments or amino acid sequence variants of native polypeptides, peptides or proteins. In some aspects, inhibition in the presence of the antagonist is observed in a dose-dependent manner. In some aspects, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower than the signal measured with a negative control under comparable conditions. Also disclosed herein, are methods of identifying antagonists suitable for use in the methods of the disclosure. For example, these methods include, but are not limited to, binding assays such as enzyme-linked immuno-absorbent assay (ELISA), ForteBio®systems, radioimmunoassay (RIA), Meso Scale Discovery assay (e.g., Meso Scale Discovery Electrochemiluminescence (MSD-ECL), and bead-based Luminex® assay. These assays determine the ability of an antagonist to bind the polypeptide of interest (e.g., a receptor or ligand) and therefore indicate the ability of the antagonist to inhibit, neutralize or block the activity of the polypeptide. Efficacy of an antagonist can also be determined using functional assays, such as the ability of an antagonist to inhibit the function of the polypeptide or an agonist. For example, a functional assay may comprise contacting a polypeptide with a candidate antagonist molecule and measuring a detectable change in one or more biological activities normally associated with the polypeptide. The potency of an antagonist is usually defined by its IC₅₀ value (concentration required to inhibit 50% of the agonist response). The lower the IC₅₀ value the greater the potency of the antagonist and the lower the concentration that is required to inhibit the maximum biological response.

As used herein, the phrase “antibody that antagonizes human IL-27, or an antigen binding portion thereof” refers to an antibody that antagonizes at least one art-recognized activity of human IL-27 (e.g., IL-27 biological activity and/or downstream pathway(s) mediated by IL-27 signaling or other IL-27-mediated function), for example, relating to a decrease (or reduction) in human IL-27 activity that is at least 5%, 10%, 20%, 30%, 40%, 50%, 60%, 70%, 80%, 90%, or more. Additional examples of IL-27 biological activities and/or downstream pathway(s) mediated by IL-27 signaling or other IL-27-mediated function are described in additional detail below and elsewhere herein.

As used herein, the term “anti-IL-27 antagonist antibody” (interchangeably termed “anti-IL-27 antibody”) refers to an antibody that specifically binds to IL-27 and inhibits IL-27 biological activity and/or downstream pathway(s) mediated by IL-27 signaling or other IL-27-mediated function. An anti-IL-27 antagonist antibody encompasses antibodies that block, antagonize, suppress, inhibit or reduce an IL-27 biological activity (e.g., ligand binding, enzymatic activity), including downstream pathways mediated by IL-27 signaling or function, such as receptor binding and/or elicitation of a cellular response to IL-27 or its metabolites. In some aspects, an anti-IL-27 antagonist antibody provided by the disclosure binds to human IL-27 and prevents, blocks, or inhibits binding of human IL-27 to its cognate or normal receptor (e.g., IL-27 receptor), or one or more receptor subunits (e.g., gp130 and/or IL-27Rα (also known as WSX1/TCCR)). In some aspects, the anti-IL-27 antagonist antibody prevents, blocks, or inhibits the binding of human IL-27 to the gp130. In some aspects, the anti-IL-27 antagonist antibody prevents, blocks, or inhibits the binding of human IL-27 to the IL-27Rα. In some aspects, the anti-IL-27 antagonist antibody prevents, blocks, or inhibits the dimerization of IL-27 monomers. In some aspects, the anti-IL-27 antibody does not specifically bind to the EBI3 monomer. In some aspects, the anti-IL-27 antibody specifically binds to the IL-27p28 monomer. In some aspects, the anti-IL-27 antibody specifically binds to a non-contiguous epitope comprising P28, but does not bind to the EBI3 monomer. In some aspects, the anti-IL-27 antibody inhibits or reduces STAT1 and/or STAT3 phosphorylation in a cell. In some aspects, the anti-IL-27 antibody inhibits or reduces inhibition of CD161 expression in a cell (e.g., ameliorates or relieves IL-27 mediated inhibition of CD161 expression in a cell). In some aspects, the anti-IL-27 antibody inhibits or reduces PD-L1 expression in a cell. In some aspects, the anti-IL-27 induces or enhances PD-1-mediated secretion of one or more cytokines from a cell. In some aspects, the anti-IL-27 antibody alters the expression of TIM-3 in a cell. In some aspects, an anti-IL-27 antagonist antibody binds to human IL-27 and stimulates or enhances an anti-tumor response. In some aspects, the anti-IL-27 antagonist antibody binds to human IL-27 with an affinity of 15 nM or less. In some aspects, the anti-IL-27 antagonist antibody binds to human IL-27 and comprises a wild type or mutant IgG1 heavy chain constant region or a wild type or mutant IgG4 heavy chain constant region. Examples of anti-IL-27 antagonist antibodies are provided herein.

As used herein, the term “antibody” refers to a whole antibody comprising two light chain polypeptides and two heavy chain polypeptides. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody. As used herein, the term “antibody fragment,” “antigen-binding fragment,” or similar terms refer to a fragment of an antibody that retains the ability to bind to a target antigen (e.g., IL-27) and inhibit the activity of the target antigen. Such fragments include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al., (2001) J. Immunol. Methods 248(1):47-66; Hudson and Kortt, (1999) J. Immunol. Methods 231(1):177-189; Poljak, (1994) Structure 2(12):1121-1123; Rondon and Marasco, (1997) Annu. Rev. Microbiol. 51:257-283, the disclosures of each of which are incorporated herein by reference in their entirety.

As used herein, the term “antibody fragment” also includes, e.g., single domain antibodies such as camelized single domain antibodies. See, e.g., Muyldermans et al., (2001) Trends Biochem. Sci. 26:230-235; Nuttall et al., (2000) Curr. Pharm. Biotech. 1:253-263; Reichmann et al., (1999) J. Immunol. Meth. 231:25-38; PCT application publication nos. WO 94/04678 and WO 94/25591; and U.S. Pat. No. 6,005,079, all of which are incorporated herein by reference in their entireties. In some aspects, the disclosure provides single domain antibodies comprising two VH domains with modifications such that single domain antibodies are formed. The precise amino acid sequence boundaries of a given CDR can be readily determined using any of a number of well-known schemes, including as described by Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme).

In some aspects, an antigen-binding fragment includes the variable region of a heavy chain polypeptide and the variable region of a light chain polypeptide. In some aspects, an antigen-binding fragment described herein comprises the CDRs of the light chain and heavy chain polypeptide of an antibody.

“Pembrolizumab,” as used herein, refers to a humanized monoclonal IgG4 (S228P) antibody directed against human cell surface receptor PD-1 (programmed death-1 or programmed cell death-1). Pembrolizumab is marketed by Merck under the name KEYTRUDA®, and it has been referred to as lambrolizumab and MK-3475. Pembrolizumab is described, for example, in U.S. Pat. Nos. 8,354,509 and 8,900,587, each of which is incorporated by reference herein in its entirety.

As used herein, the term “apoptosis” refers to the process of programmed cell death that occurs in multicellular organisms (e.g. humans). The highly regulated biochemical and molecular events that result in apoptosis can lead to observable and characteristic morphological changes to a cell, including membrane blebbing, cell volume shrinkage, chromosomal DNA condensation and fragmentation, and mRNA decay. A common method to identify cells, including T cells, undergoing apoptosis is to expose cells to a fluorophore-conjugated protein (Annexin V). Annexin V is commonly used to detect apoptotic cells by its ability to bind to phosphatidylserine on the outer leaflet of the plasma membrane, which is an early indicator that the cell is undergoing the process of apoptosis.

As used herein, the term “B cell” (alternatively “B lymphocyte”) refers to a type of white blood cell of the lymphocyte subtype. B cells function in the humoral immunity component of the adaptive immune system by secreting antibodies. B cells also present antigen and secrete cytokines. B cells, unlike the other two classes of lymphocytes, T cells and natural killer cells, express B cell receptors (BCRs) on their cell membrane. BCRs allow the B cell to bind to a specific antigen, against which it will initiate an antibody response.

As used herein, the term “binds to immobilized IL-27,” refers to the ability of an antibody of the disclosure to bind to IL-27, for example, expressed on the surface of a cell or which is attached to a solid support.

As used herein, “cancer antigen” or “tumor antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, the term “cancer-specific immune response” refers to the immune response induced by the presence of tumors, cancer cells, or cancer antigens. In certain aspects, the response includes the proliferation of cancer antigen specific lymphocytes. In certain aspects, the response includes expression and upregulation of antibodies and T-cell receptors and the formation and release of lymphokines, chemokines, and cytokines. Both innate and acquired immune systems interact to initiate antigenic responses against the tumors, cancer cells, or cancer antigens. In certain aspects, the cancer-specific immune response is a T cell response.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The anti-IL-27 antibodies described herein can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

As used herein, the term “IL-27” or “interleukin 27” refers to the IL-27 cytokine. IL-27 is related to the IL-6/IL-12 cytokine families, and is a heterodimeric cytokine that comprises a first subunit known as Epstein-Barr Virus Induced Gene 3 (EBI3; also known as IL-27 subunit β and IL-27B) and a second subunit known as IL-27p28 (also known as IL30, IL-27 subunit a and IL-27A). IL-27 is predominantly synthesized by activated antigen-presenting cells including monocytes, endothelial cells and dendritic cells (Jankowski et al. (2010) Arch Immunol. Ther. Exp. 58:417-425, Diakowski et al. (2013) Adv. Clin. Exp. Med. (2013) 22(5): 683-691). Although IL-27 can have proinflammatory effects, many studies suggest an important role of IL-27 as an immunosuppressive agent (Shimizu et al. (2006) J. Immunol. 176:7317-7324, Hisada et al. (2004) Cancer Res. 64:1152-1156, Diakowski (2013) supra). Although it was initially described as a factor promoting the initiation of Th1 responses, IL-27 was later found to play a major T-cell suppressive function by limiting Th1 responses, inhibiting Th2 and Th17 cell differentiation, and regulating the development of Tr1 and other T regulatory cell populations (Dietrich et al. (2014) J. Immunol. 192:5382-5389). In addition to its role as an immunoregulator, IL-27 also regulates angiogenesis, hematopoiesis, and osteocalstogenesis (Id.).

IL-27 signals through a heterodimeric type I cytokine receptor (the IL-27 receptor or IL-27R) that comprises a first subunit known as WSX1 (also known as IL-27 receptor subunit α, IL-27RA, T-Cell Cytokine Receptor Type 1 (TCCR), and Cytokine Receptor-Like 1 (CRL1)) and a second subunit known as gp130 (also known as Interleukin-6 Signal Transducer (IL6ST), Interleukin-6 Receptor Subunit β (IL-6RB), and Oncostatin M Receptor). gp130 is also a receptor subunit for the IL-6 family cytokines (Liu et al. (2008) Scan. J. Immunol. 68:22-299, Diakowski (2013) supra). IL-27 signaling through IL-27R activates multiple signaling cascades, including the JAK-STAT and p38 MAPK pathways.

EBI3 is also believed to have biological functions independent of p28 or the IL-27 heterodimer. For example, EBI3 also interacts with p35 to form the heterodimeric cytokine IL-35 (Yoshida et al. (2015) Annu. Rev Immunol. 33:417-43) and has been shown to be selectively overexpressed in certain cell types without a corresponding increase in p28 or IL-27 (Larousserie et al. (2005) Am. J. Pathol. 166(4):1217-28).

An amino acid sequence of an exemplary human EBI3 protein is provided in
SEQ ID NO: 1
(NCBI Reference Sequence: NP_005746.2; N-
mtpqlllalvlwascppcsgrkgppaaltlprvqcrasrypiavdcswtlppapnstspvsfiatyrlgmaarghswpclqqtptstsctit
dvqlfsmapyvlnvtavhpwgssssfvpfitehiikpdppegvrlsplaerqlqvqweppgswpfpeifslkywirykrqgaarfhrv
gpieatsfilravrpraryyvqvaaqdltdygelsdwslpatatmslgk-C).
An amino acid sequence of an exemplary human p28 protein is provided in
SEQ ID NO: 2
(NCBI Reference Sequence: NP_663634.2; N-
mgqtagdlgwrlsllllplllvqagvwgfprppgrpqlslqelrreftvslhlarkllsevrgqahrfaeshlpgvnlyllplgeqlpdvsltf
qawrrlsdperlcfisttlqpfhallgglgtqgrwtnmermqlwamrldlrdlqrhlrfqvlaagfnlpeeeeeeeeeeeeerkgllpgalg
salqgpaqvswpqllstyrllhslelvlsravrellllskaghsvwplgfptlspqp-C).
An amino acid sequence of an exemplary human WSX1 protein is provided in
SEQ ID NO: 3
(NCBI Reference Sequence: NP_004834.1; N-
mrggrgapfwlwplpklallpllwvlfqrtrpqgsagplqcygvgplgdlncsweplgdlgapselhlqsqkyrsnktqtvavaagrs
wvaipreqltmsdkllvwgtkagqplwppvfvnletqmkpnaprlgpdvdfseddpleatvhwapptwpshkvlicqfhyrrcqea
awtllepelktipltpveiqdlelatgykvygrcrmekeedlwgewspilsfqtppsapkdvwvsgnlcgtpggeeplllwkapgpcv
qvsykvwfwvggrelspegitcccslipsgaewarvsavnatswepltnlslvcldsasaprsvavssiagstellvtwqpgpgepleh
vvdwardgdpleklnwvrlppgnlsallpgnftvgvpyritvtavsasglasassvwgfreelaplvgptlwrlqdappgtpaiawgev
prhqlrghlthytlcaqsgtspsvcmnvsgntqsvtlpdlpwgpcelwvtastiagqgppgpilrlhlpdntlrwkvlpgilflwglfllgc
glslatsgrcyhlrhkvlprwvwekvpdpansssgqphmeqvpeaqplgdlpileveemepppvmessqpaqatapldsgyekhf
lptpeelgllgpprpqvla-C).
An amino acid sequence of an exemplary human gp130 protein is provided in
SEQ ID NO: 4
(NCBI Reference Sequence: NP_002175.2; N-
mltlqtwlvqalfiflttestgelldpcgyispespvvqlhsnftavcvlkekcmdyfhvnanyivwktnhftipkeqytiinrtassvtftd
iaslniqltcniltfgqleqnvygitiisglppekpknlscivnegkkmrcewdggrethletnftlksewathkfadckakrdtptsctvd
ystvyfvnievwveaenalgkvtsdhinfdpvykvkpnpphnlsvinseelssilkltwtnpsiksviilkyniqyrtkdastwsqippe
dtastrssftvqdlkpfteyvfrircmkedgkgywsdwseeasgityedrpskapsfwykidpshtqgyrtvqlvwktlppfeangkil
dyevtltrwkshlqnytvnatkltvnltndrylatltvrnlvgksdaavltipacdfqathpvmdlkafpkdnmlwvewttpresvkkyi
lewcvlsdkapcitdwqqedgtvhrtylrgnlaeskcylitvtpvyadgpgspesikaylkqappskgptvrtkkvgkneavlewdql
pvdvqngfirnytifyrtiignetavnvdsshteytlssltsdtlymvrmaaytdeggkdgpeftfttpkfaqgeieaivvpvclafllttllg
vlfcfnkrdlikkhiwpnvpdpskshiaqwsphtpprhnfnskdqmysdgnftdvsvveieandkkpfpedlksldlfkkekinteg
hssgiggsscmsssrpsisssdenessqntsstvqystvvhsgyrhqvpsvqvfsrsestqplldseerpedlqlvdhvdggdgilprqq
yfkqncsqhesspdishferskqvssvneedfvrlkqqisdhisqscgsgqmkmfqevsaadafgpgtegqverfetvgmeaatdeg
mpksylpqtvrqggympq-C).

As used herein, the term “Programmed Cell Death Protein 1” or “PD-1” refers to the Programmed Cell Death Protein 1 polypeptide, an immune-inhibitory receptor belonging to the CD28 family and is encoded by the PDCD1 gene in humans. Alternative names or synonyms for PD-1 include: PDCD1, PD1, CD279 and SLEB2. PD-1 is expressed predominantly on previously activated T cells, B cells, and myeloid cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773.

As used herein, the term “Programmed Death Ligand-1” or “PD-L1” is one of two cell surface glycoprotein ligands for PD-1 (the other being PD-L2) that downregulates T cell activation and cytokine secretion upon binding to PD-1. Alternative names and synonyms for PD-L1 include: PDCD1L1, PDL1, B7H1, B7-4, CD274 and B7-H. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

PD-1 is known as an immune-inhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to a decrease in T-cell receptor mediated proliferation (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

For several cancers, tumor survival and proliferation is sustained by tumor-mediated immune checkpoint modulation. This modulation can result in the disruption of anti-cancer immune system functions. For example, recent studies have indicated that the expression of immune checkpoint receptors ligands, such as PD-L1 or PD-L2, by tumor cells can downregulate immune system activity in the tumor microenvironment and promote cancer immune evasion. particularly by suppressing T cells. PD-L1 is abundantly expressed by a variety of human cancers (Dong et al., (2002) Nat Med 8:787-789). The receptor for PD-L1, PD-1, is expressed on lymphocytes (e.g., activated T cells) and is normally involved in down-regulating the immune system and promoting self-tolerance, particularly by suppressing T cells. However, when PD-1 receptors expressed on T cells bind to cognate PD-L1 ligands on tumor cells, the resulting T cell suppression contributes to an impaired immune response against the tumor (e.g., a decrease in tumor infiltrating lymphocytes or the establishment of immune evasion by cancer cells).

In large sample sets of e.g. ovarian, renal, colorectal, pancreatic, liver cancers and melanoma, it was shown that PD-L1 expression correlated with poor prognosis and reduced overall survival irrespective of subsequent treatment (see e.g., Dong et al., (2002) Nat Med 8(8):793-800; Yang et al., (2008) Invest Ophthalmol Vis Sci 49(6):2518-2525; Ghebeh et al., (2006) Neoplasia 8:190-198; Hamanishi et al., (2007) Proc Nat Acad Sci USA 104:3360-3365; Thompson et al., (2006) Clin Genitourin Cancer 5:206-211; Nomi et al., (2005) Clin Cancer Res 11:2947-2953; Inman et al., (2007) Cancer 109:1499-1505; Shimauchi et al., (2007) Int J Cancer 121:2585-2590; Gao et al., (2009) Clin Cancer Res 15:971-979; Nakanishi et al., (2007) Cancer Immunol Immunother 56:1173-1182; Hino et al., (2010) Cancer 116(7):1757-1766). Similarly, PD-1 expression on tumor lymphocytes was found to mark dysfunctional T cells in breast cancer (Kitano et al., (2017) ESMO Open 2(2):e000150) and melanoma (Kleffel et al., (2015) Cell 162(6):1242-1256). PD-1 antagonists, such as those that affect the function of the PD-1/PD-L1/PD-L2 signaling axis and/or disrupt the interaction between PD-1 and PD-L1 and/or PD-L2, for example, have been developed and represent a novel class of anti-tumor inhibitors that function via modulation of immune cell-tumor cell interaction. In some aspects, the PD-1 antagonist comprises pembrolizumab.

As used herein the term “compete”, when used in the context of antigen-binding proteins (e.g., immunoglobulins, antibodies, or antigen-binding fragments thereof) that compete for binding to the same epitope, refers to a interaction between antigen-binding proteins as determined by an assay (e.g., a competitive binding assay; a cross-blocking assay), wherein a test antigen-binding protein (e.g., a test antibody) inhibits (e.g., reduces or blocks) specific binding of a reference antigen-binding protein (e.g., a reference antibody; e.g., an anti-IL-27 antibody comprising a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15) to a common antigen (e.g., IL-27 or a fragment thereof).

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In certain aspects, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In certain aspects, the antibodies of the disclosure are encoded by a nucleotide sequence. Nucleotide sequences of the disclosure can be useful for a number of applications, including: cloning, gene therapy, protein expression and purification, mutation introduction, DNA vaccination of a host in need thereof, antibody generation for, e.g., passive immunization, PCR, primer and probe generation, and the like.

It will also be understood by one of ordinary skill in the art that the antibodies suitable for use in the methods disclosed herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The antibodies suitable for use in the methods disclosed herein may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain aspects, a string of amino acids can be replaced with a structurally similar string that differs in order and/or composition of side chain family members. Alternatively, in certain aspects, mutations may be introduced randomly along all or part of a coding sequence, such as by saturation mutagenesis, and the resultant mutants can be incorporated into binding polypeptides of the disclosure and screened for their ability to bind to the desired target.

As used herein, the term antigen “cross-presentation” refers to presentation of exogenous protein antigens to T cells via MHC class I and class II molecules on APCs.

As used herein, the term “cross-reacts” refers to the ability of an antibody of the disclosure to bind to IL-27 from a different species. For example, an antibody of the present disclosure which binds human IL-27 may also bind another species of IL-27. As used herein, cross-reactivity is measured by detecting a specific reactivity with purified antigen in binding assays (e.g., SPR, ELISA) or binding to, or otherwise functionally interacting with, cells physiologically expressing IL-27. Methods for determining cross-reactivity include standard binding assays as described herein, for example, by Biacore™ surface plasmon resonance (SPR) analysis using a Biacore™ 2000 SPR instrument (Biacore AB, Uppsala, Sweden), or flow cytometric techniques.

As used herein, the term “cytotoxic T lymphocyte (CTL) response” refers to an immune response induced by cytotoxic T cells. CTL responses are mediated primarily by CD8⁺ T cells.

As used herein, the term “dendritic cell” or “DC” refers to type of antigen-presenting cells that are bone marrow (BM)-derived leukocytes and are the most potent type of antigen-presenting cells. DCs are capture and process antigens, converting proteins to peptides that are presented on major histocompatibility complex (MHC) molecules recognized by T cells. DCs are heterogeneous, e.g. myeloid and plasmacytoid DCs; although all DCs are capable of antigen uptake, processing and presentation to naive T cells, the DC subtypes have distinct markers and differ in location, migratory pathways, detailed immunological function and dependence on infections or inflammatory stimuli for their generation. During the development of an adaptive immune response, the phenotype and function of DCs play a role in initiating tolerance, memory, and polarized T-helper 1 (Th1), Th2 and Th17 differentiation.

As used herein, the term “dendritic cell activation” refers to the transition from immature to mature dendritic cell; and the activated dendritic cells encompass mature dendritic cells and dendritic cells in the process of the transition, wherein the expression of CD80 and CD86 that induce costimulatory signals are elevated by the activating stimuli. Mature human dendritic cells are cells that are positive for the expression of CD40, CD80, CD86, and HLA-class II (e.g., HLA-DR). An immature dendritic cell can be distinguished from a mature dendritic cell, for example, based on markers selected from the group consisting of CD80 and CD86. An immature dendritic cell is weakly positive and preferably negative for these markers, while a mature dendritic cell is positive. Discrimination of mature dendritic cells is routinely performed by those skilled in the art, and the respective markers described above and methods for measuring their expression are also well known to those skilled in the art.

As used herein, the term “EC5o” refers to the concentration of an antibody or an antigen-binding portion thereof, which induces a response, either in an in vitro or an in vivo assay, which is 50% of the maximal response, i.e., halfway between the maximal response and the baseline.

As used herein, the term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “epitope” or “antigenic determinant” refers to a site on an antigen to which an immunoglobulin or antibody specifically binds. The term “epitope mapping” refers to a process or method of identifying the binding site, or epitope, of an antibody, or antigen binding fragment thereof, on its target protein antigen. Epitope mapping methods and techniques are provided herein. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from IL-27 are tested for reactivity with the given anti-IL-27 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

As used herein, the term “Fc-mediated effector functions” or “Fc effector functions” refer to the biological activities of an antibody other than the antibody's primary function and purpose. For example, the effector functions of a therapeutic agnostic antibody are the biological activities other than the activation of the target protein or pathway. Examples of antibody effect functions include C1q binding and complement dependent cytotoxicity; Fc receptor binding; antibody-dependent cell-mediated cytotoxicity (ADCC); phagocytosis; down regulation of cell surface receptors (e.g., B cell receptor); lack of activation of platelets that express Fc receptor; and B cell activation. Many effector functions begin with Fc binding to an Fcγ receptor. In some aspects, the tumor antigen-targeting antibody has effector function, e.g., ADCC activity. In some aspects, a tumor antigen-targeting antibody described herein comprises a variant constant region having increased effector function (e.g. increased ability to mediate ADCC) relative to the unmodified form of the constant region.

As used herein, the term “Fc receptor” refers to a polypeptide found on the surface of immune effector cells, which is bound by the Fc region of an antibody. In some aspects, the Fc receptor is an Fcγ receptor. There are three subclasses of Fcγ receptors, FcγRI (CD64), FcγRII (CD32) and FcγRIII (CD16). All four IgG isotypes (IgG1, IgG2, IgG3 and IgG4) bind and activate Fc receptors FcγRI, FcγRIIA and FcγRIIIA. FcγRIIB is an inhibitory receptor, and therefore antibody binding to this receptor does not activate complement and cellular responses. FcγRI is a high affinity receptor that binds to IgG in monomeric form, whereas FcγRIIA and FcγRIIA are low affinity receptors that bind IgG only in multimeric form and have slightly lower affinity. The binding of an antibody to an Fc receptor and/or C1q is governed by specific residues or domains within the Fc regions. Binding also depends on residues located within the hinge region and within the CH2 portion of the antibody. In some aspects, the agonistic and/or therapeutic activity of the antibodies described herein is dependent on binding of the Fc region to the Fc receptor (e.g., FcγR). In some aspects, the agonistic and/or therapeutic activity of the antibodies described herein is enhanced by binding of the Fc region to the Fc receptor (e.g., FcγR).

A list of certain Fc receptor sequences employed in the instant disclosure is set forth as Table 1B below.

As used herein, the term “glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the nonhuman transgenic animal, when one of ordinary skill in the art would recognize the glycosylation pattern of the heterologous antibody as being more similar to said pattern of glycosylation in the species of the nonhuman transgenic animal than to the species from which the CH genes of the transgene were derived.

As used herein, the term “human antibody” includes antibodies having variable and constant regions (if present) of human germline immunoglobulin sequences. Human antibodies of the disclosure can include amino acid residues not encoded by human germline immunoglobulin sequences (e.g., mutations introduced by random or site-specific mutagenesis in vitro or by somatic mutation in vivo) (See, e.g., Lonberg et al., (1994) Nature 368(6474): 856-859); Lonberg, (1994) Handbook of Experimental Pharmacology 113:49-101; Lonberg & Huszar, (1995) Intern. Rev. Immunol. 13:65-93, and Harding & Lonberg, (1995) Ann. N.Y. Acad. Sci. 764:536-546). However, the term “human antibody” does not include antibodies in which CDR sequences derived from the germline of another mammalian species, such as a mouse, have been grafted onto human framework sequences (i.e. humanized antibodies).

As used herein, the term a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The terms “inducing an immune response” and “enhancing an immune response” are used interchangeably and refer to the stimulation of an immune response (i.e., either passive or adaptive) to a particular antigen. The terms “induce” as used with respect to inducing CDC or ADCC refer to the stimulation of particular direct cell killing mechanisms.

As used herein, the term “immunogenic cell death” (alternatively known as “immunogenic apoptosis” refers to a cell death modality associated with the activation of one or more signaling pathways that induces the pre-mortem expression and emission of damaged-associated molecular pattern (DAMPs) molecules (e.g., adenosine triphosphate, ATP) from the tumor cell, resulting in the increase of immunogenicity of the tumor cell and the death of the tumor cell in an immunogenic manner (e.g., by phagocytosis). As used herein, the term “immunogenic cell death-inducing agent” refers to a chemical, biological, or pharmacological agent that induces an immunogenic cell death process, pathway, or modality.

As used herein, the terms “inhibits”, “reduces” or “blocks” (e.g., referring to inhibition or reduction of human IL-27-mediated phosphorylation of STAT1 and/or STAT3 in a cell) are used interchangeably and encompass both partial and complete inhibition/blocking. The inhibition/blocking of IL-27 and/or PD-1 reduces or alters the normal level or type of activity that occurs without inhibition or blocking. Inhibition and blocking are also intended to include any measurable decrease in the binding affinity of target protein, e.g., IL-27 or PD-1 when in contact with an antibody, e.g., an anti-IL-27 antibody or pembrolizumab, respectively, as compared to the target protein not in contact with a corresponding antibody, e.g., inhibits binding of IL-27 by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

As used herein, the terms “inhibits angiogenesis,” “diminishes angiogenesis,” and “reduces angiogenesis” refer to reducing the level of angiogenesis in a tissue to a quantity which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control tissue, and most preferably is at the same level which is observed in a control tissue.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a an anti-IL-27 antibody disclosed herein and pembrolizumab).

The term “in vivo” refers to processes that occur in a living organism.

As used herein, the term “isolated antibody” is intended to refer to an antibody which is substantially free of other antibodies having different antigenic specificities (e.g., an isolated antibody that specifically binds to human IL-27 is substantially free of antibodies that specifically bind antigens other than IL-27). An isolated antibody that specifically binds to an epitope may, however, have cross-reactivity to other proteins, e.g., other IL-27 proteins, from different species. However, the antibody continues to display specific binding to human IL-27 in a specific binding assay as described herein. In addition, an isolated antibody is typically substantially free of other cellular material and/or chemicals.

As used herein, the term “isolated nucleic acid molecule” refers to nucleic acids encoding antibodies or antibody portions (e.g., V_{H}, V_{L}, CDR3) that bind to IL-27, is intended to refer to a nucleic acid molecule in which the nucleotide sequences encoding the antibody or antibody portion are free of other nucleotide sequences encoding antibodies or antibody portions that bind antigens other than IL-27, which other sequences may naturally flank the nucleic acid in human genomic DNA.

As used herein, “isotype” refers to the antibody class (e.g., IgM or IgG1) that is encoded by heavy chain constant region genes. In some aspects, a human monoclonal antibody of the disclosure is of the IgG1 isotype. In some aspects, a human monoclonal antibody of the disclosure is of the IgG2 isotype. In some aspects, a human monoclonal antibody of the disclosure is of the IgG3 isotype. In some aspects, a human monoclonal antibody of the disclosure is of the IgG4 isotype. As is apparent to a skilled artisan, identification of antibody isotypes (e.g., IgG1, IgG2, IgG3, IgG4, IgM, IgA1 IgA2, IgD, and IgE) is routine in the art and commonly involves a combination of sequence alignments with known antibodies, published Fc variant sequences and conserved sequences.

As used herein, the term “isotype switching” refers to the phenomenon by which the class, or isotype, of an antibody changes from one Ig class to one of the other Ig classes.

As used herein the term “KD” or “K_{D}” refers to the equilibrium dissociation constant of a binding reaction between an antibody and an antigen. The value of K_{D} is a numeric representation of the ratio of the antibody off-rate constant (kd) to the antibody on-rate constant (ka). The value of K_{D} is inversely related to the binding affinity of an antibody to an antigen. The smaller the K_{D} value the greater the affinity of the antibody for its antigen. Affinity is the strength of binding of a single molecule to its ligand and is typically measured and reported by the equilibrium dissociation constant (K_{D}), which is used to evaluate and rank order strengths of bimolecular interactions.

As used herein, the term “kd” or “k_{d}” (alternatively “koff” or “k_{off}”) is intended to refer to the off-rate constant for the dissociation of an antibody from an antibody/antigen complex. The value of k_{d} is a numeric representation of the fraction of complexes that decay or dissociate per second, and is expressed in units sec⁻¹.

As used herein, the term “ka” or “kₐ” (alternatively “kon” or “kₒₙ”) is intended to refer to the on-rate constant for the association of an antibody with an antigen. The value of ka is a numeric representation of the number of antibody/antigen complexes formed per second in a 1 molar (1M) solution of antibody and antigen, and is expressed in units M⁻¹sec⁻¹.

As used herein, the term “leukocyte” refers to a type of white blood cell involved in defending the body against infective organisms and foreign substances. Leukocytes are produced in the bone marrow. There are 5 main types of white blood cells, subdivided between 2 main groups: polymorphonuclear leukocytes (neutrophils, eosinophils, basophils) and mononuclear leukocytes (monocytes and lymphocytes).

As used herein, the term “lymphocytes” refers to a type of leukocyte or white blood cell that is involved in the immune defenses of the body. There are two main types of lymphocytes: B-cells and T-cells.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, “local administration” or “local delivery,” refers to delivery that does not rely upon transport of the composition or agent to its intended target tissue or site via the vascular system. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site.

As used herein, “MHC molecules” refers to two types of molecules, MHC class I and MHC class II. MHC class I molecules present antigen to specific CD8+ T cells and MHC class II molecules present antigen to specific CD4+ T cells. Antigens delivered exogenously to APCs are processed primarily for association with MHC class II. In contrast, antigens delivered endogenously to APCs are processed primarily for association with MHC class I.

As used herein, the term “monoclonal antibody” refers to an antibody which displays a single binding specificity and affinity for a particular epitope. Accordingly, the term “human monoclonal antibody” refers to an antibody which displays a single binding specificity and which has variable and optional constant regions derived from human germline immunoglobulin sequences. In some aspects, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “monocyte” refers to a type of leukocyte and can differentiate into macrophages and dendritic cells to effect an immune response.

As used herein, the term “natural killer (NK) cell” refers to a type of cytotoxic lymphocyte. These are large, usually granular, non-T, non-B lymphocytes, which kill certain tumor cells and play an important role in innate immunity to viruses and other intracellular pathogens, as well as in antibody-dependent cell-mediated cytotoxicity (ADCC).

As used herein, the term “naturally occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

As used herein, the term “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As used herein, the term “patient” includes human and other mammalian subjects that receive either prophylactic or therapeutic treatment.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the terms “polypeptide,” “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “preventing” when used in relation to a condition, refers to administration of a composition which reduces the frequency of, or delays the onset of, symptoms of a medical condition in a subject relative to a subject which does not receive the composition.

As used herein, the term “purified” or “isolated” as applied to any of the proteins (antibodies or fragments) described herein refers to a polypeptide that has been separated or purified from components (e.g., proteins or other naturally occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

As used herein, the term “recombinant host cell” (or simply “host cell”) is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

As used herein, the terms “specific binding,” “selective binding,” “selectively binds,” and “specifically binds,” refer to antibody binding to an epitope on a predetermined antigen. Typically, the antibody binds with an equilibrium dissociation constant (K_{D}) of approximately less than 10⁻⁶ M, such as approximately less than 10⁻⁷, 10⁻⁸ M, 10⁻⁹ M or 10⁻¹⁰ M or even lower when determined by surface plasmon resonance (SPR) technology in a BIACORE 2000 instrument using recombinant human IL-27 as the analyte and the antibody as the ligand and binds to the predetermined antigen with an affinity that is at least two-fold greater than its affinity for binding to a non-specific antigen (e.g., BSA, casein) other than the predetermined antigen or a closely-related antigen. In certain aspects, an antibody that specifically binds to IL-27 binds with an equilibrium dissociation constant (K_{D}) of approximately less than 100 nM (10⁻⁷ M), optionally approximately less than 50 nM (5×10⁻¹ M), optionally approximately less than 15 nM (1.5×10⁻⁸ M), optionally approximately less than 10 nM (10⁻⁸ M), optionally approximately less than 5 nM (5×10⁻⁹ M), optionally approximately less than 1 nM (10⁻⁹ M), optionally approximately less than 0.1 nM (10⁻¹⁰ M), optionally approximately less than 0.01 nM (10⁻¹⁰ M), or even lower, when determined by surface plasmon resonance (SPR) technology in a BIACORE 2000 instrument using recombinant human IL-27 as the analyte and the antibody as the ligand, where binding to the predetermined antigen occurs with an affinity that is at least two-fold greater than the antibody's affinity for binding to a non-specific antigen (e.g., BSA, casein) other than the predetermined antigen or a closely-related antigen The phrases “an antibody recognizing an antigen” and “an antibody specific for an antigen” are used interchangeably herein with the term “an antibody which binds specifically to an antigen.”

As used herein, the term “STAT1 phosphorylation” refers to the phosphorylation of the Signal Transducer and Activator of Transcription 1 (STAT1) polypeptide, a transcription factor encoded by the STAT1 gene in humans. STAT molecules are phosphorylated by receptor associated kinases, that cause activation and dimerization by forming homo- or heterodimers which translocate to the nucleus to work as transcription factors. STAT1 can be activated (i.e., phosphorylated) in response to signaling via several ligands, including IL-27. IL-27 signaling through the IL-27R results in phosphorylation of STAT1 (pSTAT1). STAT1 has a key role in gene expression involved in survival of the cell, viability or pathogen response. Methods to determine STAT1 phosphorylation as a result of IL-27 signaling include, but are not limited to, flow cytometric analysis of cells labeled with antibodies that specifically recognize phosphorylated STAT1 (see e.g., Tochizawa et al., (2006) J Immunol Methods 313(1-2):29-37).

As used herein, the term “STAT3 phosphorylation” refers to the phosphorylation of the Signal Transducer and Activator of Transcription 3 (STAT3) polypeptide, a transcription factor encoded by the STAT3 gene in humans. STAT3 mediates the expression of a variety of genes in response to cell stimuli, and thus plays a key role in many cellular processes such as cell growth and apoptosis. Methods to determine STAT3 phosphorylation as a result of IL-27 signaling include, but are not limited to, analysis of cells or cell extracts labeled with antibodies that specifically recognize phosphorylated STAT3 (see e.g., Fursov et al., (2011) Assay Drug Dev Technol 9(4):420-429).

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., y, c, etc.). As there is no specific site where recombination always occurs, the final gene sequence will typically not be predictable from the construct.

As used herein, the term “subject” includes any human or non-human animal. For example, the methods and compositions of the present disclosure can be used to treat a subject with an immune disorder. The term “non-human animal” includes all vertebrates, e.g., mammals and non-mammals, such as non-human primates, sheep, dog, cow, chickens, amphibians, reptiles, etc.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the disclosure. BLAST protein searches can be performed with the XBLAST program, score=50, wordlength=3 to obtain amino acid sequences homologous to the protein molecules of the disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The term “T cell” refers to a type of white blood cell that can be distinguised from other white blood cells by the presence of a T cell receptor on the cell surface. There are several subsets of T cells, including, but not limited to, T helper cells (a.k.a. Tx cells or CD4⁺ T cells) and subtypes, including T^{H}1, T^{H}2, T^{H}3, T^{H}17, T^{H}9, and T^{FH} cells, cytotoxic T cells (a.k.a T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺FOXP3⁻ T_{reg} cells, Tr1 cells, Th3 cells, and T_{reg} 17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the disclosure.

As used herein, the term “T cell-mediated response” refers to any response mediated by T cells, including, but not limited to, effector T cells (e.g., CD8⁺ cells) and helper T cells (e.g., CD4⁺ cells). T cell mediated responses include, for example, T cell cytotoxicity and proliferation.

As used herein, the terms “therapeutically effective amount” or “therapeutically effective dose,” or similar terms used herein are intended to mean an amount of an agent (e.g., an anti-IL-27 antibody or an antigen-binding fragment thereof) that will elicit the desired biological or medical response (e.g., an improvement in one or more symptoms of a cancer).

As used herein, the term “TAM receptor” refers to the TAM receptor protein tyrosine kinases (TYRO3, AXL and MER). TAM receptors are involved in the regulation of immune system homeostasis. In a cancer setting, TAM receptors have a dual regulatory role, controlling the initiation and progression of tumor development and, at the same time, the associated anti-tumor responses of diverse immune cells. Further description of TAM receptors is found in Paolino and Penninger (2016) Cancers 8(97): doi:10.3390/cancers8100097). As used herein, the term “TAM receptor inhibitor” or “TAM inhibitor” refers to an agent that inhibits, blocks or reduces the function or activity of a TAM receptor.

As used herein, the term “TIGIT” or “T-cell immunoreceptor with Ig and ITIM domains” refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term also encompasses naturally occurring variants of TIGIT, e.g., splice variants or allelic variants. The amino acid sequence of an exemplary human TIGIT may be found under UniProt Accession Number Q495A1.

The terms “treat,” “treating,” and “treatment,” as used herein, refer to therapeutic or preventative measures described herein. The methods of “treatment” employ administration to a subject, in need of such treatment, a human antibody of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

As used herein, the term “tumor microenvironment” (alternatively “cancer microenvironment”; abbreviated TME) refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the TME. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

### II. Methods of the Disclosure

Some aspects of the present disclosure are directed to methods of stimulating an immune response in a subject, the method comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15. Some aspects of the present disclosure are directed to methods of treating a cancer in a subject in need thereof comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

The method described herein can be used in the treatment of various types of cancer. In some aspects, the cancer is selected from Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma (RCC), hepatocellular carcinoma (HCC), hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, retinoblastoma, rhabdomyosarcoma, rectal cancer; cancer of the respiratory system, sarcoma, skin cancer, stomach cancer, testicular cancer, thyroid cancer, uterine cancer, cancer of the urinary system, and any combination thereof. In some aspects, the cancer comprises lung cancer (e.g., non-small cell lung cancer), sarcoma, testicular cancer, ovarian cancer, pancreas cancer, breast cancer (e.g., triple-negative breast cancer), melanoma, head and neck cancer (e.g., squamous head and neck cancer), colorectal cancer, bladder cancer, endometrial cancer, prostate cancer, thyroid cancer, hepatocellular carcinoma, gastric cancer, brain cancer, lymphoma (e.g., DL-BCL), leukemia (e.g., AML), renal cancer (e.g., renal cell carcinoma, e.g., clear cell RCC and/or non-clear cell RCC), or any combination thereof. In some aspects, the cancer comprises a non-small cell lung cancer (NSCLC). In some aspects, the cancer comprises a hepatocellular carcinoma (HCC). In some aspects, the cancer comprises a renal cancer. In some aspects, the renal cancer is a renal cell carcinoma (RCC). In some aspects, the RCC is a clear cell RCC. In some aspects, the RCC is a non-clear cell RCC.

In some aspects, the methods can be performed in conjunction with other therapies for cancer. For example, the composition can be administered to a subject at the same time, prior to, or after, radiation, surgery, targeted or cytotoxic chemotherapy, chemoradiotherapy, hormone therapy, immunotherapy, gene therapy, cell transplant therapy, precision medicine, genome editing therapy, or other pharmacotherapy.

In some aspects, the methods disclosed herein comprise administering an anti-IL-27 antibody disclosed herein, pembrolizumab, and a chemotherapeutic agent. Chemotherapeutic agents suitable for combination and/or co-administration with compositions of the present disclosure include, for example: taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g., mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II)(DDP), procarbazine, altretamine, cisplatin, carboplatin, oxaliplatin, nedaplatin, satraplatin, or triplatin tetranitrate), anthracycline (e.g., daunorubicin (formerly daunomycin) and doxorubicin), antibiotics (e.g., dactinomcin (formerly actinomycin), bleomycin, mithramycin, and anthramycin (AMC)), and anti-mitotic agents (e.g., vincristine and vinblastine) and temozolomide.

In some aspects, the compositions disclosed herein are administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), or intrathecal injection (IT). The injection can be in a bolus or a continuous infusion.

Administration can be achieved by, e.g., local infusion, injection, or by means of an implant. The implant can be of a porous, non-porous, or gelatinous material, including membranes, such as silastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some aspects, the anti-IL-27 antibody and/or pembrolizumab is therapeutically delivered to a subject by way of local administration.

In certain aspects, the route of administration is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain aspects, the compositions can be administered by bolus injection or continuously by infusion, or by implantation device. In certain aspects, individual elements of the combination therapy may be administered by different routes.

In certain aspects, the anti-IL-27 antibody and/or pembrolizumab is administered locally via implantation of a membrane, sponge or another appropriate material onto which the desired molecule has been absorbed or encapsulated. In certain aspects, where an implantation device is used, the device is implanted into any suitable tissue or organ, and delivery of the desired molecule can be via diffusion, timed-release bolus, or continuous administration. In certain aspects, it is desirable to use a pharmaceutical composition comprising the anti-IL-27 antibody and/or pembrolizumab in an ex vivo manner. In such instances, cells, tissues and/or organs that have been removed from the patient are exposed to a pharmaceutical composition comprising the anti-IL-27 antibody and/or pembrolizumab after which the cells, tissues and/or organs are subsequently implanted back into the patient.

In certain aspects, the anti-IL-27 antibody and/or pembrolizumab is delivered by implanting certain cells that have been genetically engineered, using methods such as those described herein, to express and secrete the polypeptides. In certain aspects, such cells are animal or human cells, and can be autologous, heterologous, or xenogeneic. In certain aspects, the cells are immortalized. In certain aspects, in order to decrease the chance of an immunological response, the cells are encapsulated to avoid infiltration of surrounding tissues. In certain aspects, the encapsulation materials are typically biocompatible, semi-permeable polymeric enclosures or membranes that allow the release of the protein product(s) but prevent the destruction of the cells by the patient's immune system or by other detrimental factors from the surrounding tissues.

In some aspects, following administration of the anti-IL-27 antibody and/or pembrolizumab, the subject exhibits increased expression of one or more biomarkers selected from the group consisting of Eotaxin-1 (CCL11), TARC (CCL17), VEGF-A, IL-7, IL-8, MCP-1, MCP-4, and any combination thereof; wherein the increased expression of the one or more biomarkers is relative to the expression of the one or more biomarker prior to the administration. In some aspects, following administration antibody or antigen binding portion thereof, the subject exhibits increased expression of Eotaxin-1 (CCL11), wherein the increased expression of Eotaxin-1 (CCL11) is relative to the expression of Eotaxin-1 (CCL11) prior to the administration.

In some aspects, following administration of the anti-IL-27 antibody and/or pembrolizumab, the subject exhibits increased expression of TARC (CCL17), wherein the increased expression of TARC (CCL17) is relative to the expression of TARC (CCL17) prior to the administration.

In some aspects, following administration of the anti-IL-27 antibody and/or pembrolizumab, the subject exhibits increased expression of VEGF-A, wherein the increased expression of VEGF-A is relative to the expression of VEGF-A prior to the administration.

In some aspects, following administration of the anti-IL-27 antibody and/or pembrolizumab, the subject exhibits increased expression of IL-7, wherein the increased expression of IL-7 is relative to the expression of IL-7 prior to the administration.

In some aspects, following administration of the anti-IL-27 antibody and/or pembrolizumab, the subject exhibits increased expression of IL-8, wherein the increased expression of IL-8 is relative to the expression of IL-8 prior to the administration.

In some aspects, following administration of the anti-IL-27 antibody and/or pembrolizumab, the subject exhibits increased expression of MCP-1, wherein the increased expression of MCP-1 is relative to the expression of MCP-1 prior to the administration.

In some aspects, following administration of the anti-IL-27 antibody and/or pembrolizumab, the subject exhibits increased expression of MCP-4, wherein the increased expression of MCP-4 is relative to the expression of MCP-4 prior to the administration.

### A. Anti-IL-27 Antibodies and Antigen-Binding Portions Thereof

In some aspects, the anti-IL-27 antibody comprising a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15 is administered at a dose of at least about 0.003 mg/kg to at least about 20 mg/kg. In some aspects, the anti-IL-27 antibody is administered at a dose that is sufficient to maintain IC90 of pSTAT1 inhibition level, i.e., above about 0.7 ug/mL for the duration of the treatment, e.g., 28 days, 56 days, or 84 days.

In some aspects, the anti-IL-27 antibody is administered at a dose of at least about 0.006 mg/kg to at least about 20 mg/kg, at least about 0.009 mg/kg to at least about 20 mg/kg, at least about 0.01 mg/kg to at least about 20 mg/kg, at least about 0.03 mg/kg to at least about 20 mg/kg, at least about 0.06 mg/kg to at least about 20 mg/kg, at least about 0.09 mg/kg to at least about 20 mg/kg, at least about 0.1 mg/kg to at least about 20 mg/kg, at least about 0.3 mg/kg to at least about 20 mg/kg, at least about 0.6 mg/kg to at least about 20 mg/kg, at least about 0.9 mg/kg to at least about 20 mg/kg, at least about 1 mg/kg to at least about 20 mg/kg, at least about 1 mg/kg to at least about 20 mg/kg, at least about 3 mg/kg to at least about 20 mg/kg, at least about 6 mg/kg to at least about 20 mg/kg, at least about 10 mg/kg to at least about 20 mg/kg, at least about 13 mg/kg to at least about 20 mg/kg, at least about 13 mg/kg to at least about 18 mg/kg, at least about 13 mg/kg to at least about 16 mg/kg, at least about 16 mg/kg to at least about 20 mg/kg, at least about 16 mg/kg to at least about 18 mg/kg, at least about 3 mg/kg to at least about 18 mg/kg, at least about 6 mg/kg to at least about 15 mg/kg, at least about 13 mg/kg to at least about 18 mg/kg, or at least about 10 mg/kg to at least about 15 mg/kg. In some aspects, the antibody or antigen binding portion thereof is administered at a dose of at least about 0.006 mg/kg to at least about 10 mg/kg, at least about 0.009 mg/kg to at least about 10 mg/kg, at least about 0.01 mg/kg to at least about 10 mg/kg, at least about 0.03 mg/kg to at least about 10 mg/kg, at least about 0.06 mg/kg to at least about 10 mg/kg, at least about 0.09 mg/kg to at least about 10 mg/kg, at least about 0.1 mg/kg to at least about 10 mg/kg, at least about 0.3 mg/kg to at least about 10 mg/kg, at least about 0.6 mg/kg to at least about 10 mg/kg, at least about 0.9 mg/kg to at least about 10 mg/kg, at least about 1 mg/kg to at least about 10 mg/kg, at least about 1 mg/kg to at least about 9 mg/kg, at least about 3 mg/kg to at least about 9 mg/kg, at least about 1 mg/kg to at least about 6 mg/kg, at least about 3 mg/kg to at least about 6 mg/kg, or at least about 1 mg/kg to at least about 3 mg/kg.

In some aspects, the antibody or antigen binding portion thereof is administered at a dose of at least about 0.003 mg/kg, at least about 0.006 mg/kg, at least about 0.009 mg/kg, at least about 0.01 mg/kg, at least about 0.03 mg/kg, at least about 0.06 mg/kg, at least about 0.09 mg/kg, at least about 0.1 mg/kg, at least about 0.3 mg/kg, at least about 0.6 mg/kg, at least about 0.9 mg/kg, at least about 1.0 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9, or at least about 10 mg/kg. In some aspects, the antibody or antigen binding portion thereof is administered at a dose of at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19, or at least about 20 mg/kg. In some aspects, the antibody or antigen binding portion thereof is administered at a dose of at least about 0.003 mg/kg. In some aspects, the antibody or antigen binding portion thereof is administered at a dose of at least about 0.006 mg/kg. In some aspects, the antibody or antigen binding portion thereof is administered at a dose of at least about 0.009 mg/kg. In some aspects, the antibody or antigen binding portion thereof is administered at a dose of at least about 0.01 mg/kg. In some aspects, the antibody or antigen binding portion thereof is administered at a dose of at least about 0.03 mg/kg. In some aspects, the antibody or antigen binding portion thereof is administered at a dose of at least about 0.06 mg/kg. In some aspects, the antibody or antigen binding portion thereof is administered at a dose of at least about 0.09 mg/kg. In some aspects, the antibody or antigen binding portion thereof is administered at a dose of at least about 0.1 mg/kg. In some aspects, the antibody or antigen binding portion thereof is administered at a dose of at least about 0.3 mg/kg. In some aspects, the antibody or antigen binding portion thereof is administered at a dose of at least about 0.6 mg/kg. In some aspects, the antibody or antigen binding portion thereof is administered at a dose of at least about 0.9 mg/kg. In some aspects, the antibody or antigen binding portion thereof is administered at a dose of at least about 1.0 mg/kg. In some aspects, the antibody or antigen binding portion thereof is administered at a dose of at least about 2 mg/kg. In some aspects, the antibody or antigen binding portion thereof is administered at a dose of at least about 3 mg/kg. In some aspects, the antibody or antigen binding portion thereof is administered at a dose of at least about 4 mg/kg. In some aspects, the antibody or antigen binding portion thereof is administered at a dose of at least about 5 mg/kg. In some aspects, the antibody or antigen binding portion thereof is administered at a dose of at least about 6 mg/kg. In some aspects, the antibody or antigen binding portion thereof is administered at a dose of at least about 7 mg/kg. In some aspects, the antibody or antigen binding portion thereof is administered at a dose of at least about 8 mg/kg. In some aspects, the antibody or antigen binding portion thereof is administered at a dose of at least about 9 mg/kg. In some aspects, the antibody or antigen binding portion thereof is administered at a dose of at least about 10 mg/kg. In some aspects, the antibody or antigen binding portion thereof is administered at a dose of at least about 11 mg/kg. In some aspects, the antibody or antigen binding portion thereof is administered at a dose of at least about 12 mg/kg. In some aspects, the antibody or antigen binding portion thereof is administered at a dose of at least about 13 mg/kg. In some aspects, the antibody or antigen binding portion thereof is administered at a dose of at least about 14 mg/kg. In some aspects, the antibody or antigen binding portion thereof is administered at a dose of at least about 15 mg/kg. In some aspects, the antibody or antigen binding portion thereof is administered at a dose of at least about 16 mg/kg. In some aspects, the antibody or antigen binding portion thereof is administered at a dose of at least about 17 mg/kg. In some aspects, the antibody or antigen binding portion thereof is administered at a dose of at least about 18 mg/kg. In some aspects, the antibody or antigen binding portion thereof is administered at a dose of at least about 19 mg/kg. In some aspects, the antibody or antigen binding portion thereof is administered at a dose of at least about 20 mg/kg.

In some aspects, the antibody or antigen binding portion thereof is administered once about every week, once about every two weeks, once about every three weeks, once about every four weeks, once about every five weeks, once about every 6 weeks, once about every 7 weeks, once about every 8 weeks, or once about every 12 weeks. In some aspects, the antibody or antigen binding portion thereof is administered once about every two weeks. In some aspects, the antibody or antigen binding portion thereof is administered once about every three weeks. In some aspects, the antibody or antigen binding portion thereof is administered once about every four weeks.

In some aspects, the antibody or antigen binding portion thereof is administered at a dose of about 0.3 mg/kg once about every week. In some aspects, the antibody or antigen binding portion thereof is administered at a dose of about 1 mg/kg once about every week. In some aspects, the antibody or antigen binding portion thereof is administered at a dose of about 2 mg/kg once about every week. In some aspects, the antibody or antigen binding portion thereof is administered at a dose of about 3 mg/kg once about every week. In some aspects, the antibody or antigen binding portion thereof is administered at a dose of about 4 mg/kg once about every week. In some aspects, the antibody or antigen binding portion thereof is administered at a dose of about 5 mg/kg once about every week. In some aspects, the antibody or antigen binding portion thereof is administered at a dose of about 6 mg/kg once about every week. In some aspects, the antibody or antigen binding portion thereof is administered at a dose of about 7 mg/kg once about every week. In some aspects, the antibody or antigen binding portion thereof is administered at a dose of about 8 mg/kg once about every week. In some aspects, the antibody or antigen binding portion thereof is administered at a dose of about 9 mg/kg once about every week. In some aspects, the antibody or antigen binding portion thereof is administered at a dose of about 10 mg/kg once about every week. In some aspects, the antibody or antigen binding portion thereof is administered at a dose of about 11 mg/kg once about every week. In some aspects, the antibody or antigen binding portion thereof is administered at a dose of about 12 mg/kg once about every week. In some aspects, the antibody or antigen binding portion thereof is administered at a dose of about 13 mg/kg once about every week. In some aspects, the antibody or antigen binding portion thereof is administered at a dose of about 14 mg/kg once about every week. In some aspects, the antibody or antigen binding portion thereof is administered at a dose of about 15 mg/kg once about every week. In some aspects, the antibody or antigen binding portion thereof is administered at a dose of about 16 mg/kg once about every week. In some aspects, the antibody or antigen binding portion thereof is administered at a dose of about 17 mg/kg once about every week. In some aspects, the antibody or antigen binding portion thereof is administered at a dose of about 18 mg/kg once about every week. In some aspects, the antibody or antigen binding portion thereof is administered at a dose of about 19 mg/kg once about every week. In some aspects, the antibody or antigen binding portion thereof is administered at a dose of about 20 mg/kg once about every week.

In some aspects, the antibody or antigen binding portion thereof is administered at a dose of about 0.3 mg/kg once about every two weeks. In some aspects, the antibody or antigen binding portion thereof is administered at a dose of about 1 mg/kg once about every two weeks. In some aspects, the antibody or antigen binding portion thereof is administered at a dose of about 2 mg/kg once about every two weeks. In some aspects, the antibody or antigen binding portion thereof is administered at a dose of about 3 mg/kg once about every two weeks. In some aspects, the antibody or antigen binding portion thereof is administered at a dose of about 4 mg/kg once about every two weeks. In some aspects, the antibody or antigen binding portion thereof is administered at a dose of about 5 mg/kg once about every two weeks. In some aspects, the antibody or antigen binding portion thereof is administered at a dose of about 6 mg/kg once about every two weeks. In some aspects, the antibody or antigen binding portion thereof is administered at a dose of about 7 mg/kg once about every two weeks. In some aspects, the antibody or antigen binding portion thereof is administered at a dose of about 8 mg/kg once about every two weeks. In some aspects, the antibody or antigen binding portion thereof is administered at a dose of about 9 mg/kg once about every two weeks. In some aspects, the antibody or antigen binding portion thereof is administered at a dose of about 10 mg/kg once about every two weeks. In some aspects, the antibody or antigen binding portion thereof is administered at a dose of about 11 mg/kg once about every two weeks. In some aspects, the antibody or antigen binding portion thereof is administered at a dose of about 12 mg/kg once about every two weeks. In some aspects, the antibody or antigen binding portion thereof is administered at a dose of about 13 mg/kg once about every two weeks. In some aspects, the antibody or antigen binding portion thereof is administered at a dose of about 14 mg/kg once about every two weeks. In some aspects, the antibody or antigen binding portion thereof is administered at a dose of about 15 mg/kg once about every two weeks. In some aspects, the antibody or antigen binding portion thereof is administered at a dose of about 16 mg/kg once about every two weeks. In some aspects, the antibody or antigen binding portion thereof is administered at a dose of about 17 mg/kg once about every two weeks. In some aspects, the antibody or antigen binding portion thereof is administered at a dose of about 18 mg/kg once about every two weeks. In some aspects, the antibody or antigen binding portion thereof is administered at a dose of about 19 mg/kg once about every two weeks. In some aspects, the antibody or antigen binding portion thereof is administered at a dose of about 20 mg/kg once about every two weeks.

In some aspects, the antibody or antigen binding portion thereof is administered at a dose of about 0.3 mg/kg once about every three weeks. In some aspects, the antibody or antigen binding portion thereof is administered at a dose of about 1 mg/kg once about every three weeks. In some aspects, the antibody or antigen binding portion thereof is administered at a dose of about 2 mg/kg once about every three weeks. In some aspects, the antibody or antigen binding portion thereof is administered at a dose of about 3 mg/kg once about every three weeks. In some aspects, the antibody or antigen binding portion thereof is administered at a dose of about 4 mg/kg once about every three weeks. In some aspects, the antibody or antigen binding portion thereof is administered at a dose of about 5 mg/kg once about every three weeks. In some aspects, the antibody or antigen binding portion thereof is administered at a dose of about 6 mg/kg once about every three weeks. In some aspects, the antibody or antigen binding portion thereof is administered at a dose of about 7 mg/kg once about every three weeks. In some aspects, the antibody or antigen binding portion thereof is administered at a dose of about 8 mg/kg once about every three weeks. In some aspects, the antibody or antigen binding portion thereof is administered at a dose of about 9 mg/kg once about every three weeks. In some aspects, the antibody or antigen binding portion thereof is administered at a dose of about 10 mg/kg once about every three weeks. In some aspects, the antibody or antigen binding portion thereof is administered at a dose of about 11 mg/kg once about every three weeks. In some aspects, the antibody or antigen binding portion thereof is administered at a dose of about 12 mg/kg once about every three weeks. In some aspects, the antibody or antigen binding portion thereof is administered at a dose of about 13 mg/kg once about every three weeks. In some aspects, the antibody or antigen binding portion thereof is administered at a dose of about 14 mg/kg once about every three weeks. In some aspects, the antibody or antigen binding portion thereof is administered at a dose of about 15 mg/kg once about every three weeks. In some aspects, the antibody or antigen binding portion thereof is administered at a dose of about 16 mg/kg once about every three weeks. In some aspects, the antibody or antigen binding portion thereof is administered at a dose of about 17 mg/kg once about every three weeks. In some aspects, the antibody or antigen binding portion thereof is administered at a dose of about 18 mg/kg once about every three weeks. In some aspects, the antibody or antigen binding portion thereof is administered at a dose of about 19 mg/kg once about every three weeks. In some aspects, the antibody or antigen binding portion thereof is administered at a dose of about 20 mg/kg once about every three weeks.

In some aspects, the antibody or antigen binding portion thereof is administered at a dose of about 0.3 mg/kg once about every four weeks. In some aspects, the antibody or antigen binding portion thereof is administered at a dose of about 1 mg/kg once about every four weeks. In some aspects, the antibody or antigen binding portion thereof is administered at a dose of about 2 mg/kg once about every four weeks. In some aspects, the antibody or antigen binding portion thereof is administered at a dose of about 3 mg/kg once about every four weeks. In some aspects, the antibody or antigen binding portion thereof is administered at a dose of about 4 mg/kg once about every four weeks. In some aspects, the antibody or antigen binding portion thereof is administered at a dose of about 5 mg/kg once about every four weeks. In some aspects, the antibody or antigen binding portion thereof is administered at a dose of about 6 mg/kg once about every four weeks. In some aspects, the antibody or antigen binding portion thereof is administered at a dose of about 7 mg/kg once about every four weeks. In some aspects, the antibody or antigen binding portion thereof is administered at a dose of about 8 mg/kg once about every four weeks. In some aspects, the antibody or antigen binding portion thereof is administered at a dose of about 9 mg/kg once about every four weeks. In some aspects, the antibody or antigen binding portion thereof is administered at a dose of about 10 mg/kg once about every four weeks. In some aspects, the antibody or antigen binding portion thereof is administered at a dose of about 11 mg/kg once about every four weeks. In some aspects, the antibody or antigen binding portion thereof is administered at a dose of about 12 mg/kg once about every four weeks. In some aspects, the antibody or antigen binding portion thereof is administered at a dose of about 13 mg/kg once about every four weeks. In some aspects, the antibody or antigen binding portion thereof is administered at a dose of about 14 mg/kg once about every four weeks. In some aspects, the antibody or antigen binding portion thereof is administered at a dose of about 15 mg/kg once about every four weeks. In some aspects, the antibody or antigen binding portion thereof is administered at a dose of about 16 mg/kg once about every four weeks. In some aspects, the antibody or antigen binding portion thereof is administered at a dose of about 17 mg/kg once about every four weeks. In some aspects, the antibody or antigen binding portion thereof is administered at a dose of about 18 mg/kg once about every four weeks. In some aspects, the antibody or antigen binding portion thereof is administered at a dose of about 19 mg/kg once about every four weeks. In some aspects, the antibody or antigen binding portion thereof is administered at a dose of about 20 mg/kg once about every four weeks.

In some aspects, the antibody or antigen binding portion thereof is administered at a dose of about 0.3 mg/kg once about every six weeks. In some aspects, the antibody or antigen binding portion thereof is administered at a dose of about 1 mg/kg once about every six weeks. In some aspects, the antibody or antigen binding portion thereof is administered at a dose of about 2 mg/kg once about every six weeks. In some aspects, the antibody or antigen binding portion thereof is administered at a dose of about 3 mg/kg once about every six weeks. In some aspects, the antibody or antigen binding portion thereof is administered at a dose of about 4 mg/kg once about every six weeks. In some aspects, the antibody or antigen binding portion thereof is administered at a dose of about 5 mg/kg once about every six weeks. In some aspects, the antibody or antigen binding portion thereof is administered at a dose of about 6 mg/kg once about every six weeks. In some aspects, the antibody or antigen binding portion thereof is administered at a dose of about 7 mg/kg once about every six weeks. In some aspects, the antibody or antigen binding portion thereof is administered at a dose of about 8 mg/kg once about every six weeks. In some aspects, the antibody or antigen binding portion thereof is administered at a dose of about 9 mg/kg once about every six weeks. In some aspects, the antibody or antigen binding portion thereof is administered at a dose of about 10 mg/kg once about every six weeks. In some aspects, the antibody or antigen binding portion thereof is administered at a dose of about 11 mg/kg once about every six weeks. In some aspects, the antibody or antigen binding portion thereof is administered at a dose of about 12 mg/kg once about every six weeks. In some aspects, the antibody or antigen binding portion thereof is administered at a dose of about 13 mg/kg once about every six weeks. In some aspects, the antibody or antigen binding portion thereof is administered at a dose of about 14 mg/kg once about every six weeks. In some aspects, the antibody or antigen binding portion thereof is administered at a dose of about 15 mg/kg once about every six weeks. In some aspects, the antibody or antigen binding portion thereof is administered at a dose of about 16 mg/kg once about every six weeks. In some aspects, the antibody or antigen binding portion thereof is administered at a dose of about 17 mg/kg once about every six weeks. In some aspects, the antibody or antigen binding portion thereof is administered at a dose of about 18 mg/kg once about every six weeks. In some aspects, the antibody or antigen binding portion thereof is administered at a dose of about 19 mg/kg once about every six weeks. In some aspects, the antibody or antigen binding portion thereof is administered at a dose of about 20 mg/kg once about every six weeks.

In some aspects, the anti-IL-27 antibody or antigen binding portion thereof disclosed herein (i.e., comprising a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15) inhibits or reduces STAT1 and/or STAT3 phosphorylation in a cell in the subject. In some aspects, the anti-IL-27 antibody or antigen binding portion thereof inhibits or reduces pSTAT1 signaling (e.g., IL-27 mediated pSTAT1 signaling). In some aspects, the anti-IL-27 antibody or antigen binding portion thereof inhibits or reduces pSTAT1 signaling (e.g., IL-27 mediated pSTAT1 signaling) by at least about 25%, at least about 50%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, relative to pSTAT1 signaling (e.g., IL-27 mediated pSTAT1 signaling) prior to administration of the antibody or antigen binding portion thereof (e.g., an anti-IL-27 antibody disclosed herein). In some aspects, the anti-IL-27 antibody or antigen binding portion thereof inhibits or reduces PSTAT1 signaling (e.g., IL-27 mediated pSTAT1 signaling) by at least about 90% relative to PSTAT1 signaling (e.g., IL-27 mediated pSTAT1 signaling) prior to administration of the antibody or antigen binding portion thereof (e.g., an anti-IL-27 antibody disclosed herein). In some aspects, the anti-IL-27 antibody or antigen binding portion thereof inhibits or reduces PSTAT1 signaling (e.g., IL-27 mediated pSTAT1 signaling) by at least about 91% relative to PSTAT1 signaling (e.g., IL-27 mediated pSTAT1 signaling) prior to administration of the anti-IL-27 antibody or antigen binding portion thereof. In some aspects, the anti-IL-27 antibody or antigen binding portion thereof inhibits or reduces PSTAT1 signaling (e.g., IL-27 mediated pSTAT1 signaling) by at least about 92% relative to PSTAT1 signaling (e.g., IL-27 mediated pSTAT1 signaling) prior to administration of the anti-IL-27 antibody or antigen binding portion thereof. In some aspects, the anti-IL-27 antibody or antigen binding portion thereof inhibits or reduces PSTAT1 signaling (e.g., IL-27 mediated pSTAT1 signaling) by at least about 93% relative to PSTAT1 signaling (e.g., IL-27 mediated pSTAT1 signaling) prior to administration of the anti-IL-27 antibody or antigen binding portion thereof. In some aspects, the anti-IL-27 antibody or antigen binding portion thereof inhibits or reduces PSTAT1 signaling (e.g., IL-27 mediated pSTAT1 signaling) by at least about 94% relative to PSTAT1 signaling (e.g., IL-27 mediated pSTAT1 signaling) prior to administration of the anti-IL-27 antibody or antigen binding portion thereof. In some aspects, the anti-IL-27 antibody or antigen binding portion thereof inhibits or reduces PSTAT1 signaling (e.g., IL-27 mediated pSTAT1 signaling) by at least about 95% relative to PSTAT1 signaling (e.g., IL-27 mediated pSTAT1 signaling) prior to administration of the anti-IL-27 antibody or antigen binding portion thereof. In some aspects, the anti-IL-27 antibody or antigen binding portion thereof inhibits or reduces PSTAT1 signaling (e.g., IL-27 mediated pSTAT1 signaling) by at least about 96% relative to PSTAT1 signaling (e.g., IL-27 mediated pSTAT1 signaling) prior to administration of the anti-IL-27 antibody or antigen binding portion thereof (e.g., an anti-IL-27 antibody disclosed herein). In some aspects, the anti-IL-27 antibody or antigen binding portion thereof inhibits or reduces PSTAT1 signaling (e.g., IL-27 mediated pSTAT1 signaling) by at least about 97% relative to PSTAT1 signaling (e.g., IL-27 mediated pSTAT1 signaling) prior to administration of the anti-IL-27 antibody or antigen binding portion thereof. In some aspects, the anti-IL-27 antibody or antigen binding portion thereof inhibits or reduces PSTAT1 signaling (e.g., IL-27 mediated pSTAT1 signaling) by at least about 98% relative to PSTAT1 signaling (e.g., IL-27 mediated pSTAT1 signaling) prior to administration of the anti-IL-27 antibody or antigen binding portion thereof. In some aspects, the anti-IL-27 antibody or antigen binding portion thereof inhibits or reduces PSTAT1 signaling (e.g., IL-27 mediated pSTAT1 signaling) by at least about 99% relative to PSTAT1 signaling (e.g., IL-27 mediated pSTAT1 signaling) prior to administration of the anti-IL-27 antibody or antigen binding portion thereof.

In some aspects, the anti-IL-27 antibody or antigen binding portion thereof inhibits or reduces inhibition of CD161 expression in a cell in the subject. In some aspects, the anti-IL-27 antibody or antigen binding portion thereof inhibits or reduces PD-L1 expression in a cell in the subject. In some aspects, the anti-IL-27 antibody or antigen binding portion thereof induces or enhances PD-1 mediated secretion of one or more cytokines from a cell in the subject. In some aspects, the anti-IL-27 antibody or antigen binding portion thereof alters TIM-3 expression in a cell in the subject. In some aspects, the cell is a tumor cell or an immune cell.

Accordingly, in one aspect, the anti-IL-27 antibody or an antigen binding portion thereof exhibits at least one or more of the following properties: (i) binds to human IL-27 with an equilibrium dissociation constant (K_{D}) of 15 nM or less; (ii) blocks binding of IL-27 to IL-27 receptor; (iii) inhibits or reduces STAT1 and/or STAT3 phosphorylation in a cell; (iv) inhibits or reduces IL-27 mediated inhibition of CD161 expression in a cell; (v) inhibits or reduces IL-27 mediated PD-L1 expression in a cell; (vi) induces or enhances PD-1 mediated secretion of one or more cytokines from a cell; (vii) alters TIM-3 expression in a cell; and (viii) a combination of (i)-(vii).

In some aspects, the antibody or the antigen binding portion thereof comprises a heavy chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 11. In some aspects, the antibody or the antigen binding portion thereof comprises a heavy chain variable region comprising the amino acid sequence set forth in SEQ ID NO: 11.

In some aspects, the antibody or the antigen binding portion thereof comprises a light chain variable region comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 19. In some aspects, the antibody or the antigen binding portion thereof comprises a light chain variable region comprising the amino acid sequence set forth in SEQ ID NO: 19.

In some aspects, the antibody or the antigen binding portion thereof comprises a heavy chain variable region comprising the amino acid sequence set forth in SEQ ID NO: 11 and a light chain variable region comprising the amino acid sequence set forth in SEQ ID NO: 19.

In some aspects, the method comprises administering (i) pembrolizumab and (ii) a dose of at least about 0.003 mg/kg to at least about 20 mg/kg of an antibody or antigen binding portion thereof comprising a heavy chain variable region comprising the amino acid sequence set forth in SEQ ID NO: 11 and a light chain variable region comprising the amino acid sequence set forth in SEQ ID NO: 19. In some aspects, the method comprises administering (i) pembrolizumab and (ii) a dose of at least about 1 mg/kg of an antibody or antigen binding portion thereof comprising a heavy chain variable region comprising the amino acid sequence set forth in SEQ ID NO: 11 and a light chain variable region comprising the amino acid sequence set forth in SEQ ID NO: 19. In some aspects, the method comprises administering (i) pembrolizumab and (ii) a dose of at least about 3 mg/kg of an antibody or antigen binding portion thereof comprising a heavy chain variable region comprising the amino acid sequence set forth in SEQ ID NO: 11 and a light chain variable region comprising the amino acid sequence set forth in SEQ ID NO: 19. In some aspects, the method comprises administering (i) pembrolizumab and (ii) a dose of at least about 6 mg/kg of an antibody or antigen binding portion thereof comprising a heavy chain variable region comprising the amino acid sequence set forth in SEQ ID NO: 11 and a light chain variable region comprising the amino acid sequence set forth in SEQ ID NO: 19. In some aspects, the method comprises administering (i) pembrolizumab and (ii) a dose of at least about 10 mg/kg of an antibody or antigen binding portion thereof comprising a heavy chain variable region comprising the amino acid sequence set forth in SEQ ID NO: 11 and a light chain variable region comprising the amino acid sequence set forth in SEQ ID NO: 19. In some aspects, the method comprises (i) pembrolizumab and (ii) administering a dose of at least about 20 mg/kg of an antibody or antigen binding portion thereof comprising a heavy chain variable region comprising the amino acid sequence set forth in SEQ ID NO: 11 and a light chain variable region comprising the amino acid sequence set forth in SEQ ID NO: 19.

In some aspects, the anti-IL-27 antibody or the antigen binding portion thereof comprises a heavy chain comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 21. In some aspects, the anti-IL-27 antibody or the antigen binding portion thereof comprises a heavy chain comprising the amino acid sequence set forth in SEQ ID NO: 21.

In some aspects, the anti-IL-27 antibody or the antigen binding portion thereof comprises a heavy chain comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 25. In some aspects, the anti-IL-27 antibody or the antigen binding portion thereof comprises a heavy chain comprising the amino acid sequence set forth in SEQ ID NO: 25.

In some aspects, the anti-IL-27 antibody or the antigen binding portion thereof comprises a light chain comprising an amino acid sequence that has at least about 85%, at least about 90%, at least about 95%, at least about 96%, at least about 97%, at least about 98%, or at least about 99% sequence identity to the amino acid sequence set forth in SEQ ID NO: 23. In some aspects, the anti-IL-27 antibody or the antigen binding portion thereof comprises a light chain comprising the amino acid sequence set forth in SEQ ID NO: 23.

In some aspects, the anti-IL-27 antibody or the antigen binding portion thereof comprises a heavy chain comprising the amino acid sequence set forth in SEQ ID NO: 21 and a light chain comprising the amino acid sequence set forth in SEQ ID NO: 23. In some aspects, the method comprises administering (i) pembrolizumab and (ii) a dose of at least about 0.003 mg/kg to at least about 20 mg/kg of an antibody or antigen binding portion thereof comprising a heavy chain variable region comprising the amino acid sequence set forth in SEQ ID NO: 21 and a light chain variable region comprising the amino acid sequence set forth in SEQ ID NO: 23. In some aspects, the method comprises administering (i) pembrolizumab and (ii) a dose of at least about 1 mg/kg of an antibody or antigen binding portion thereof comprising a heavy chain variable region comprising the amino acid sequence set forth in SEQ ID NO: 21 and a light chain variable region comprising the amino acid sequence set forth in SEQ ID NO: 23. In some aspects, the method comprises administering (i) pembrolizumab and (ii) a dose of at least about 3 mg/kg of an antibody or antigen binding portion thereof comprising a heavy chain variable region comprising the amino acid sequence set forth in SEQ ID NO: 21 and a light chain variable region comprising the amino acid sequence set forth in SEQ ID NO: 23. In some aspects, the method comprises administering (i) pembrolizumab and (ii) a dose of at least about 6 mg/kg of an antibody or antigen binding portion thereof comprising a heavy chain variable region comprising the amino acid sequence set forth in SEQ ID NO: 21 and a light chain variable region comprising the amino acid sequence set forth in SEQ ID NO: 23. In some aspects, the method comprises administering (i) pembrolizumab and (ii) a dose of at least about 10 mg/kg of an antibody or antigen binding portion thereof comprising a heavy chain variable region comprising the amino acid sequence set forth in SEQ ID NO: 21 and a light chain variable region comprising the amino acid sequence set forth in SEQ ID NO: 23. In some aspects, the method comprises administering (i) pembrolizumab and (ii) a dose of at least about 20 mg/kg of an antibody or antigen binding portion thereof comprising a heavy chain variable region comprising the amino acid sequence set forth in SEQ ID NO: 21 and a light chain variable region comprising the amino acid sequence set forth in SEQ ID NO: 23.

In some aspects, the anti-IL-27 antibody or the antigen binding portion thereof comprises a heavy chain comprising the amino acid sequence set forth in SEQ ID NO: 25 and a light chain comprising the amino acid sequence set forth in SEQ ID NO: 23. In some aspects, the method comprises administering (i) pembrolizumab and (ii) a dose of at least about 0.003 mg/kg to at least about 20 mg/kg of an antibody or antigen binding portion thereof comprising a heavy chain variable region comprising the amino acid sequence set forth in SEQ ID NO: 25 and a light chain variable region comprising the amino acid sequence set forth in SEQ ID NO: 23. In some aspects, the method comprises administering (i) pembrolizumab and (ii) a dose of at least about 1 mg/kg of an antibody or antigen binding portion thereof comprising a heavy chain variable region comprising the amino acid sequence set forth in SEQ ID NO: 25 and a light chain variable region comprising the amino acid sequence set forth in SEQ ID NO: 23. In some aspects, the method comprises administering (i) pembrolizumab and (ii) a dose of at least about 3 mg/kg of an antibody or antigen binding portion thereof comprising a heavy chain variable region comprising the amino acid sequence set forth in SEQ ID NO: 25 and a light chain variable region comprising the amino acid sequence set forth in SEQ ID NO: 23. In some aspects, the method comprises administering (i) pembrolizumab and (ii) a dose of at least about 6 mg/kg of an antibody or antigen binding portion thereof comprising a heavy chain variable region comprising the amino acid sequence set forth in SEQ ID NO: 25 and a light chain variable region comprising the amino acid sequence set forth in SEQ ID NO: 23. In some aspects, the method comprises administering (i) pembrolizumab and (ii) a dose of at least about 10 mg/kg of an antibody or antigen binding portion thereof comprising a heavy chain variable region comprising the amino acid sequence set forth in SEQ ID NO: 25 and a light chain variable region comprising the amino acid sequence set forth in SEQ ID NO: 23. In some aspects, the method comprises administering (i) pembrolizumab and (ii) a dose of at least about 20 mg/kg of an antibody or antigen binding portion thereof comprising a heavy chain variable region comprising the amino acid sequence set forth in SEQ ID NO: 25 and a light chain variable region comprising the amino acid sequence set forth in SEQ ID NO: 23.

In some aspects, the anti-IL-27 antibody or antigen-binding portion thereof comprises an amino acid sequence set forth in Table 1A.

TABLE 1A
Anti-IL-27 Antibodies

| SEQ | | |
|---|---|---|
| ID NO | Description | Sequence |
| 5 | HCDR1 | GFTFRSYG |
| | (IMGT) | |
| 6 | HCDR2 | ISSSGSYI |
| | (IMGT) | |
| 7 | HCDR3 | ARDGGRTSYTATAHNWEDP |
| | (IMGT) | |
| 8 | HCDR1 (NT) | FTFRSYGMN |
| 9 | HCDR2 (NT) | GISSSGSYIYYADSVKG |
| 10 | HCDR3 (NT) | ARDGGRTSYTATAHNWFDP |
| 11 | VH | EVQLVESGGGLVKPGGSLRLSCAASGFTERSYGMNWVRQAPGKG |
| | | LEWVSGISSSGSYIYYADSVKGRFTISRDNAKNSLYLQMNSLRA |
| | | EDTAVYYCARDGGRTSYTATAHNWEDPWGQGTLVTVSS |
| 12 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 13 | LCDR1 | QSVLESSNNKNY |
| | (IMGT) | |
| 14 | LCDR2 | WAS |
| | (IMGT) | |
| 15 | LCDR3 | QQHASAPPT |
| | (IMGT) | |
| 16 | LCDR1 (NT) | KSSQSVLESSNNKNYLA |
| 17 | LCDR2 (NT) | WASTRES |
| 18 | LCDR3 (NT) | QQHASAPPT |
| 19 | VL | DIVMTQSPDSLAVSLGERATINCKSSQSVLESSNNKNYLAWYQQ |
| | | KPGQPPKLLIYWASTRESGVPDRESGSGSGTDFTLTISSLQAED |
| | | VAVYYCQQHASAPPTFGGGTKVEIK |
| 20 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 21 | Heavy Chain | EVQLVESGGGLVKPGGSLRLSCAASGFTFRSYGMNWVRQAPGKG |
| | | LEWVSGISSSGSYIYYADSVKGRFTISRDNAKNSLYLQMNSLRA |
| | | EDTAVYYCARDGGRTSYTATAHNWEDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKENWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 22 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 23 | Light Chain | DIVMTQSPDSLAVSLGERATINCKSSQSVLFSSNNKNYLAWYQQ |
| | | KPGQPPKLLIYWASTRESGVPDRESGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSENRGEC |
| 24 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |
| 25 | Heavy Chain | EVQLVESGGGLVKPGGSLRLSCAASGFTFRSYGMNWVRQAPGKG |
| | | LEWVSGISSSGSYIYYADSVKGRFTISRDNAKNSLYLQMNSLRA |
| | | EDTAVYYCARDGGRTSYTATAHNWEDPWGQGTLVTVSSASTKGP |
| | | SVEPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 26 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |

In some aspects, the anti-IL-27 antibody, or antigen binding portion thereof, comprises an Fc sequence set forth in Table 1B. In some aspects, the anti-IL-27 antibody, or antigen binding portion thereof, comprises a heavy chain, wherein the heavy chain comprises an Fc region having an amino acid at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 27, 28, 29, or 30. In some aspects, the anti-IL-27 antibody, or antigen binding portion thereof, comprises a heavy chain, wherein the heavy chain comprises an Fc region comprising the amino acid sequence set forth in SEQ ID NO: 27. In some aspects, the anti-IL-27 antibody, or antigen binding portion thereof, comprises a heavy chain, wherein the heavy chain comprises an Fc region comprising the amino acid sequence set forth in SEQ ID NO: 28. In some aspects, the anti-IL-27 antibody, or antigen binding portion thereof, comprises a heavy chain, wherein the heavy chain comprises an Fc region comprising the amino acid sequence set forth in SEQ ID NO: 29. In some aspects, the anti-IL-27 antibody, or antigen binding portion thereof, comprises a heavy chain, wherein the heavy chain comprises an Fc region comprising the amino acid sequence set forth in SEQ ID NO: 30.

TABLE 1B
Fc Sequences (═CH2 + CH3)

| Name | Alias | Amino Acid Sequence |
|---|---|---|
| Human | 1.0 | EPKSCDKTHTCPPCPAPELLGGPSVFLFPPKPKDTLMISRTPEVTC |
| IgG1 | | VVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPP |
| | | SRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSP |
| | | GK (SEQ ID NO: 27) |
| Human | 4.0 | ESKYGPPCPSCPAPEFLGGPSVFLFPPKPKDTLMISRTPEVTCVVV |
| IgG4 | | DVSQEDPEVQFNWYVDGVEVHNAKTKPREEQFNSTYRVVSVLTVLH |
| | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| | | (SEQ ID NO: 28) |
| Human | 4.1 | ESKYGPPCPPCPAPEFLGGPSVFLFPPKPKDTLMISRTPEVTCVVV |
| IgG4 | | DVSQEDPEVQFNWYVDGVEVHNAKTKPREEQFNSTYRVVSVLTVLH |
| (S228P) | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDG |
| | | SFFLYSRLTVDKSRWQEGNVESCSVMHEALHNHYTQKSLSLSLGK |
| | | (SEQ ID NO: 29) |
| Human | 4.2 | ESKYGPPCPPCPAPEFEGGPSVFLFPPKPKDTLMISRTPEVTCVVV |
| IgG4 | | DVSQEDPEVQFNWYVDGVEVHNAKTKPREEQFNSTYRVVSVLTVLH |
| (S228P/ | | QDWLNGKEYKCKVSNKGLPSSIEKTISKAKGQPREPQVYTLPPSQE |
| L235E) | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| | | (SEQ ID NO: 30) |

In some aspects, the anti-IL-27 antibody, or antigen binding portion thereof, inhibits or reduces STAT1 phosphorylation in a cell. In some aspects, the anti-IL-27 antibody, or antigen binding portion thereof, inhibits or reduces STAT3 phosphorylation in a cell. In some aspects, the anti-IL-27 antibody, or antigen binding portion thereof, inhibits or reduces STAT1 and STAT3 phosphorylation in a cell. In some aspects, the anti-IL-27 antibody, or antigen binding portion thereof, inhibits or reduces STAT1 phosphorylation in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 50%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 60%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 70%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 75%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 80%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 85%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 90%, relative to the STAT1 phosphorylation in the cell prior to contacting the cell with the antibody, or antigen binding portion thereof. In some aspects, the anti-IL-27 antibody, or antigen binding portion thereof, inhibits or reduces STAT1 phosphorylation in a cell by at least about 95%, relative to the STAT1 phosphorylation in the cell prior to contacting the cell with the antibody, or antigen binding portion thereof. In some aspects, the anti-IL-27 antibody, or antigen binding portion thereof, eliminates STAT1 phosphorylation in the cell.

In some aspects, the anti-IL-27 antibody, or antigen binding portion thereof, inhibits or reduces STAT3 phosphorylation in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 50%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 60%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 70%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 75%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 80%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 85%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 90%, relative to the STAT3 phosphorylation in the cell prior to contacting the cell with the anti-IL-27 antibody, or antigen binding portion thereof. In some aspects, the anti-IL-27 antibody, or antigen binding portion thereof, inhibits or reduces STAT3 phosphorylation in a cell by at least about 95%, relative to the STAT3 phosphorylation in the cell prior to contacting the cell with the anti-IL-27 antibody, or antigen binding portion thereof. In some aspects, the anti-IL-27 antibody, or antigen binding portion thereof, eliminates STAT3 phosphorylation in the cell.

In some aspects, the cell is an immune cell. In some aspects, the cell is a cancer cell.

In some aspects, the anti-IL-27 antibody, or antigen binding portion thereof, inhibits or reduces inhibition of CD161 expression in a cell (e.g. ameliorates or relieves the inhibition of CD161 expression in a cell). In some aspects, the cell is an immune cell.

In some aspects, the anti-IL-27 antibody, or antigen binding portion thereof, inhibits or reduces PD-L1 expression in a cell. In some aspects, PD-L1 expression is inhibited or reduced. In some aspects, TIM-3 expression is altered. In some aspects, both PD-L1 expression and TIM-3 expression is altered. In some aspects, the cell is an immune cell. In some aspects, the antibodies are monoclonal antibodies.

In some aspects, the anti-IL-27 antibody, or antigen binding portion thereof, induces or enhances the PD-1-mediated secretion of one or more cytokines from a cell. In some aspects, the one or more cytokines is TNFα. In some aspects, the one or more cytokine is IL-6. In some aspects, the one or more cytokine is TNFα and IL-6. In some aspects, the cell is an immune cell.

In some aspects, the anti-IL-27 antibody, or antigen binding portion thereof, is selected from the group consisting of an IgG1, an IgG2, an IgG3, an IgG4, an IgM, an IgA1 an IgA2, an IgD, and an IgE antibody. In some aspects, the anti-IL-27 antibody is an IgG1 antibody or an IgG4 antibody. In some aspects, the anti-IL-27 antibody comprises a wild type IgG1 heavy chain constant region. In some aspects, the anti-IL-27 antibody comprises a wild type IgG4 heavy chain constant region. In some aspects, the anti-IL-27 antibody comprises an Fc domain comprising at least one mutation. In some aspects, the anti-IL-27 antibody comprises a mutant IgG1 heavy chain constant region. In some aspects, the anti-IL-27 antibody comprises a mutant IgG4 heavy chain constant region. In some aspects, the mutant IgG4 heavy chain constant region comprises any one of the substitutions S228P, L235E, L235A, or a combination thereof, according to EU numbering.

### B. Pembrolizumab

Some aspects of the present disclosure are directed to methods of stimulating an immune response in a subject, the method comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

Pembrolizumab (also known as MK-3475) is a humanized IgG4 mAb with the structure described in WHO Drug Information, Vol. 27, No. 2, pages 161-162 (2013) and which comprises a heavy chain comprising the amino acid sequence set forth in SEQ ID NO: 39 and a light chain comprising the amino acid sequence set forth in SEQ ID NO: 40. Pembrolizumab comprises (i) a HC-CDR1 comprising the amino acid sequence set forth in SEQ ID NO: 31; (ii) a HC-CDR2 comprising the amino acid sequence set forth in SEQ ID NO: 32; (iii) a HC-CDR3 comprising the amino acid sequence set forth in SEQ ID NO: 33. (iv) a LC-CDR1 comprising the amino acid sequence set forth in SEQ ID NO: 34; (v) a LC-CDR2 comprising the amino acid sequence set forth in SEQ ID NO: 35; and (vi) a LC-CDR3 comprising the amino acid sequence set forth in SEQ ID NO: 36. Pembrolizumab comprises a variable heavy domain comprising the amino acid sequence set forth in SEQ ID NO: 37 and a variable light domain comprising the amino acid sequence set forth in SEQ ID NO: 38.

TABLE 2
Pembrolizumab Sequences

| SEQ | | |
|---|---|---|
| ID NO | Description | Sequence |
| 31 | HC-CDR1 | NYYMY |
| 32 | HC-CDR2 | GINPSNGGTNFNEKFKN |
| 33 | HC-CDR3 | RDYRFDMGFDY |
| 34 | LC-CDR1 | RASKGVSTSGYSYLH |
| 35 | LC-CDR2 | LASYLES |
| 36 | LC-CDR3 | QHSRDLPLT |
| 37 | VH | QVQLVQSGVEVKKPGASVKVSCKASGYTFTNYYMYWVR |
| | | QAPGQGLEWMGGINPSNGGTNFNEKFKNRVTLTTDSSTTT |
| | | AYMELKSLQFDDTAVYYCARRDYRFDMGFDYWGQGTTV |
| | | TVSS |
| 38 | VL | EIVLTQSPATLSLSPGERATLSCRASKGVSTSGYSYLHWYQ |
| | | QKPGQAPRLLIYLASYLESGVPARFSGSGSGTDFTLTISSLEP |
| | | EDFAVYYCQHSRDLPLTFGGGTKVEIK |
| 39 | Heavy Chain | QVQLVQSGVEVKKPGASVKVSCKASGYTFTNYYMYWVR |
| | | QAPGQGLEWMGGINPSNGGTNFNEKFKNRVTLTTDSSTTT |
| | | AYMELKSLQFDDTAVYYCARRDYRFDMGFDYWGQGTTV |
| | | TVSSASTKGPSVFPLAPCSRSTSESTAALGCLVKDYFPEPVT |
| | | VSWNSGALTSGVHTFPAVLQSSGLYSLSSVVTVPSSSLGTK |
| | | TYTCNVDHKPSNTKVDKRVESKYGPPCPPCPAPEFLGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQK |
| | | SLSLSLGK |
| 40 | Light Chain | EIVLTQSPATLSLSPGERATLSCRASKGVSTSGYSYLHWYQ |
| | | QKPGQAPRLLIYLASYLESGVPARFSGSGSGTDFTLTISSLEP |
| | | EDFAVYYCQHSRDLPLTFGGGTKVEIKRTVAAPSVFIFPPSD |
| | | EQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |

In some aspects, pembrolizumab is administered at a dose of at least about 0.1 mg/kg to at least about 20 mg/kg. In some aspects, pembrolizumab is administered at a dose of at least about 1 mg/kg to at least about 10 mg/kg. In some aspects, pembrolizumab is administered at a dose of at least about 1 mg/kg to at least about 9 mg/kg, at least about 1 mg/kg to at least about 8 mg/kg, at least about 1 mg/kg to at least about 7 mg/kg, at least about 1 mg/kg to at least about 6 mg/kg, at least about 1 mg/kg to at least about 5 mg/kg, at least about 1 mg/kg to at least about 4 mg/kg, at least about 1 mg/kg to at least about 3 mg/kg, at least about 2 mg/kg to at least about 10 mg/kg, at least about 3 mg/kg to at least about 10 mg/kg, at least about 4 mg/kg to at least about 10 mg/kg, at least about 5 mg/kg to at least about 10 mg/kg, at least about 6 mg/kg to at least about 10 mg/kg, at least about 7 mg/kg to at least about 10 mg/kg, at least about 8 mg/kg to at least about 10 mg/kg, at least about 2 mg/kg to at least about 6 mg/kg, at least about 5 mg/kg to at least about 5 mg/kg, at least about 2 mg/kg to at least about 4 mg/kg, at least about 3 mg/kg to at least about 6 mg/kg, or at least about 3 mg/kg to at least about 5 mg/kg.

In some aspects, pembrolizumab is administered at a dose of at least about 1 mg/kg, at least about 2 mg/kg, at least about 3 mg/kg, at least about 4 mg/kg once, at least about 5 mg/kg, at least about 6 mg/kg, at least about 7 mg/kg, at least about 8 mg/kg, at least about 9 mg/kg, or at least about 10 mg/kg. In some aspects, pembrolizumab is administered at a dose of about 2 mg/kg. In some aspects, pembrolizumab is administered at a dose of about 3 mg/kg. In some aspects, pembrolizumab is administered at a dose of about 4 mg/kg.

In some aspects, pembrolizumab is administered at a dose of about 1 mg/kg once about every two weeks, about 2 mg/kg once about every two weeks, about 3 mg/kg once about every two weeks, about 4 mg/kg once about every two weeks, about 5 mg/kg once about every two weeks, about 6 mg/kg once about every two weeks, about 7 mg/kg once about every two weeks, about 8 mg/kg once about every two weeks, about 9 mg/kg once about every two weeks, or about 10 mg/kg once about every two weeks. In some aspects, pembrolizumab is administered at a dose of about 2 mg/kg once about every two weeks. In some aspects, pembrolizumab is administered at a dose of about 3 mg/kg once about every two weeks. In some aspects, pembrolizumab is administered at a dose of about 4 mg/kg once about every two weeks.

In some aspects, pembrolizumab is administered at a dose of about 1 mg/kg once about every three weeks, about 2 mg/kg once about every three weeks, about 3 mg/kg once about every three weeks, about 4 mg/kg once about every three weeks, about 5 mg/kg once about every three weeks, about 6 mg/kg once about every three weeks, about 7 mg/kg once about every three weeks, about 8 mg/kg once about every three weeks, about 9 mg/kg once about every three weeks, or about 10 mg/kg once about every three weeks. In some aspects, pembrolizumab is administered at a dose of about 2 mg/kg once about every three weeks. In some aspects, pembrolizumab is administered at a dose of about 3 mg/kg once about every three weeks. In some aspects, pembrolizumab is administered at a dose of about 4 mg/kg once about every three weeks.

In some aspects, pembrolizumab is administered at a dose of about 1 mg/kg once about every four weeks, about 2 mg/kg once about every four weeks, about 3 mg/kg once about every four weeks, about 4 mg/kg once about every four weeks, about 5 mg/kg once about every four weeks, about 6 mg/kg once about every four weeks, about 7 mg/kg once about every four weeks, about 8 mg/kg once about every four weeks, about 9 mg/kg once about every four weeks, or about 10 mg/kg once about every four weeks. In some aspects, pembrolizumab is administered at a dose of about 2 mg/kg once about every four weeks. In some aspects, pembrolizumab is administered at a dose of about 3 mg/kg once about every four weeks. In some aspects, pembrolizumab is administered at a dose of about 4 mg/kg once about every four weeks.

In some aspects, pembrolizumab is administered at a dose of about 1 mg/kg once about every six weeks, about 2 mg/kg once about every six weeks, about 3 mg/kg once about every six weeks, about 4 mg/kg once about every six weeks, about 5 mg/kg once about every six weeks, about 6 mg/kg once about every six weeks, about 7 mg/kg once about every six weeks, about 8 mg/kg once about every six weeks, about 9 mg/kg once about every six weeks, or about 10 mg/kg once about every six weeks. In some aspects, pembrolizumab is administered at a dose of about 2 mg/kg once about every six weeks. In some aspects, pembrolizumab is administered at a dose of about 3 mg/kg once about every six weeks. In some aspects, pembrolizumab is administered at a dose of about 4 mg/kg once about every six weeks. In some aspects, pembrolizumab is administered at a dose of about 5 mg/kg once about every six weeks. In some aspects, pembrolizumab is administered at a dose of about 6 mg/kg once about every six weeks.

In some aspects, pembrolizumab is administered at a flat-dose, i.e., at a constant dose regardless of the weight of the subject. In some aspects, pembrolizumab is administered at a flat dose of about 200 mg. In some aspects, pembrolizumab is administered at a flat dose of about 400 mg. In some aspects, pembrolizumab is administered at a flat dose of about 200-400 mg.

In some aspects, pembrolizumab is administered at a flat-dose of about 200 mg once about every two weeks. In some aspects, pembrolizumab is administered at a flat-dose of about 200 mg once about every three weeks. In some aspects, pembrolizumab is administered at a flat-dose of about 200 mg once about every four weeks. In some aspects, pembrolizumab is administered at a flat-dose of about 200 mg once about every five weeks. In some aspects, pembrolizumab is administered at a flat-dose of about 200 mg once about every six weeks.

In some aspects, pembrolizumab is administered at a flat-dose of about 200-400 mg once about every two weeks. In some aspects, pembrolizumab is administered at a flat-dose of about 200-400 mg once about every three weeks. In some aspects, pembrolizumab is administered at a flat-dose of about 300 mg once about every four weeks. In some aspects, pembrolizumab is administered at a flat-dose of about 200-400 mg once about every five weeks. In some aspects, pembrolizumab is administered at a flat-dose of about 200-400 mg once about every six weeks.

In some aspects, pembrolizumab is administered at a flat-dose of about 400 mg once about every two weeks. In some aspects, pembrolizumab is administered at a flat-dose of about 400 mg once about every three weeks. In some aspects, pembrolizumab is administered at a flat-dose of about 400 mg once about every four weeks. In some aspects, pembrolizumab is administered at a flat-dose of about 400 mg once about every five weeks. In some aspects, pembrolizumab is administered at a flat-dose of about 400 mg once about every six weeks.

In some aspects, pembrolizumab is provided as a liquid medicament, which comprises 25 mg/ml pembrolizumab, 7% (w/v) sucrose, 0.02% (w/v) polysorbate 80 in 10 mM histidine buffer pH 5.5.

In some aspects, the selected dose of pembrolizumab is administered by IV infusion over a time period of between 25 and 40 minutes, or about 30 minutes.

In some aspects, (i) the anti-IL-27 antibody is administered at a dose of about 0.3 mg/kg once about every three weeks; and (ii) pembrolizumab is administered at a flat dose of about 200 mg once about every two weeks. In some aspects, (i) the anti-IL-27 antibody is administered at a dose of about 0.3 mg/kg once about every three weeks; and (ii) pembrolizumab is administered at a flat dose of about 200 mg once about every three weeks. In some aspects, (i) the anti-IL-27 antibody is administered at a dose of about 0.3 mg/kg once about every three weeks; and (ii) pembrolizumab is administered at a flat dose of about 200-400 mg once about every three weeks. In some aspects, (i) the anti-IL-27 antibody is administered at a dose of about 0.3 mg/kg once about every three weeks; and (ii) pembrolizumab is administered at a flat dose of about 200-400 mg once about every four weeks. In some aspects, (i) the anti-IL-27 antibody is administered at a dose of about 0.3 mg/kg once about every three weeks; and (ii) pembrolizumab is administered at a flat dose of about 200-400 mg once about every six weeks. In some aspects, (i) the anti-IL-27 antibody is administered at a dose of about 0.3 mg/kg once about every three weeks; and (ii) pembrolizumab is administered at a flat dose of about 400 mg/kg once about every four weeks. In some aspects, (i) the anti-IL-27 antibody is administered at a dose of about 0.3 mg/kg once about every three weeks; and (ii) pembrolizumab is administered at a flat dose of about 400 mg once about every six weeks.

In some aspects, (i) the anti-IL-27 antibody is administered at a dose of about 0.6 mg/kg once about every three weeks; and (ii) pembrolizumab is administered at a flat dose of about 200 mg once about every two weeks. In some aspects, (i) the anti-IL-27 antibody is administered at a dose of about 0.6 mg/kg once about every three weeks; and (ii) pembrolizumab is administered at a flat dose of about 200 mg once about every three weeks. In some aspects, (i) the anti-IL-27 antibody is administered at a dose of about 0.6 mg/kg once about every three weeks; and (ii) pembrolizumab is administered at a flat dose of about 200-400 mg once about every three weeks. In some aspects, (i) the anti-IL-27 antibody is administered at a dose of about 0.6 mg/kg once about every three weeks; and (ii) pembrolizumab is administered at a flat dose of about 200-400 mg once about every four weeks. In some aspects, (i) the anti-IL-27 antibody is administered at a dose of about 0.6 mg/kg once about every three weeks; and (ii) pembrolizumab is administered at a flat dose of about 200-400 mg once about every six weeks. In some aspects, (i) the anti-IL-27 antibody is administered at a dose of about 0.6 mg/kg once about every three weeks; and (ii) pembrolizumab is administered at a flat dose of about 400 mg once about every four weeks. In some aspects, (i) the anti-IL-27 antibody is administered at a dose of about 0.6 mg/kg once about every three weeks; and (ii) pembrolizumab is administered at a flat dose of about 400 mg once about every six weeks.

In some aspects, (i) the anti-IL-27 antibody is administered at a dose of about 1 mg/kg once about every three weeks; and (ii) pembrolizumab is administered at a flat dose of about 200 mg once about every two weeks. In some aspects, (i) the anti-IL-27 antibody is administered at a dose of about 1 mg/kg once about every three weeks; and (ii) pembrolizumab is administered at a flat dose of about 200 mg once about every three weeks. In some aspects, (i) the anti-IL-27 antibody is administered at a dose of about 1 mg/kg once about every three weeks; and (ii) pembrolizumab is administered at a flat dose of about 200-400 mg once about every three weeks. In some aspects, (i) the anti-IL-27 antibody is administered at a dose of about 1 mg/kg once about every three weeks; and (ii) pembrolizumab is administered at a flat dose of about 200-400 mg once about every four weeks. In some aspects, (i) the anti-IL-27 antibody is administered at a dose of about 1 mg/kg once about every three weeks; and (ii) pembrolizumab is administered at a flat dose of about 200-400 mg once about every six weeks. In some aspects, (i) the anti-IL-27 antibody is administered at a dose of about 1 mg/kg once about every three weeks; and (ii) pembrolizumab is administered at a flat dose of about 400 mg/kg once about every four weeks. In some aspects, (i) the anti-IL-27 antibody is administered at a dose of about 1 mg/kg once about every three weeks; and (ii) pembrolizumab is administered at a flat dose of about 400 mg once about every six weeks.

In some aspects, (i) the anti-IL-27 antibody is administered at a dose of about 2 mg/kg once about every three weeks; and (ii) pembrolizumab is administered at a flat dose of about 200 mg once about every two weeks. In some aspects, (i) the anti-IL-27 antibody is administered at a dose of about 2 mg/kg once about every three weeks; and (ii) pembrolizumab is administered at a flat dose of about 200 mg once about every three weeks. In some aspects, (i) the anti-IL-27 antibody is administered at a dose of about 2 mg/kg once about every three weeks; and (ii) pembrolizumab is administered at a flat dose of about 200-400 mg once about every three weeks. In some aspects, (i) the anti-IL-27 antibody is administered at a dose of about 2 mg/kg once about every three weeks; and (ii) pembrolizumab is administered at a flat dose of about 200-400 mg once about every four weeks. In some aspects, (i) the anti-IL-27 antibody is administered at a dose of about 2 mg/kg once about every three weeks; and (ii) pembrolizumab is administered at a flat dose of about 200-400 mg once about every six weeks. In some aspects, (i) the anti-IL-27 antibody is administered at a dose of about 2 mg/kg once about every three weeks; and (ii) pembrolizumab is administered at a flat dose of about 400 mg once about every four weeks. In some aspects, (i) the anti-IL-27 antibody is administered at a dose of about 2 mg/kg once about every three weeks; and (ii) pembrolizumab is administered at a flat dose of about 400 mg once about every six weeks.

In some aspects, (i) the anti-IL-27 antibody is administered at a dose of about 3 mg/kg once about every three weeks; and (ii) pembrolizumab is administered at a flat dose of about 200 mg once about every two weeks. In some aspects, (i) the anti-IL-27 antibody is administered at a dose of about 3 mg/kg once about every three weeks; and (ii) pembrolizumab is administered at a flat dose of about 200 mg once about every three weeks. In some aspects, (i) the anti-IL-27 antibody is administered at a dose of about 3 mg/kg once about every three weeks; and (ii) pembrolizumab is administered at a flat dose of about 200-400 mg once about every three weeks. In some aspects, (i) the anti-IL-27 antibody is administered at a dose of about 3 mg/kg once about every three weeks; and (ii) pembrolizumab is administered at a flat dose of about 200-400 mg once about every four weeks. In some aspects, (i) the anti-IL-27 antibody is administered at a dose of about 3 mg/kg once about every three weeks; and (ii) pembrolizumab is administered at a flat dose of about 200-400 mg once about every six weeks. In some aspects, (i) the anti-IL-27 antibody is administered at a dose of about 3 mg/kg once about every three weeks; and (ii) pembrolizumab is administered at a flat dose of about 400 mg/kg once about every four weeks. In some aspects, (i) the anti-IL-27 antibody is administered at a dose of about 3 mg/kg once about every three weeks; and (ii) pembrolizumab is administered at a flat dose of about 400 mg once about every six weeks.

In some aspects, (i) the anti-IL-27 antibody is administered at a dose of about 4 mg/kg once about every three weeks; and (ii) pembrolizumab is administered at a flat dose of about 200 mg once about every two weeks. In some aspects, (i) the anti-IL-27 antibody is administered at a dose of about 4 mg/kg once about every three weeks; and (ii) pembrolizumab is administered at a flat dose of about 200 mg once about every three weeks. In some aspects, (i) the anti-IL-27 antibody is administered at a dose of about 4 mg/kg once about every three weeks; and (ii) pembrolizumab is administered at a flat dose of about 200-400 mg once about every three weeks. In some aspects, (i) the anti-IL-27 antibody is administered at a dose of about 4 mg/kg once about every three weeks; and (ii) pembrolizumab is administered at a flat dose of about 200-400 mg once about every four weeks. In some aspects, (i) the anti-IL-27 antibody is administered at a dose of about 4 mg/kg once about every three weeks; and (ii) pembrolizumab is administered at a flat dose of about 200-400 mg once about every six weeks. In some aspects, (i) the anti-IL-27 antibody is administered at a dose of about 4 mg/kg once about every three weeks; and (ii) pembrolizumab is administered at a flat dose of about 400 mg once about every four weeks. In some aspects, (i) the anti-IL-27 antibody is administered at a dose of about 4 mg/kg once about every three weeks; and (ii) pembrolizumab is administered at a flat dose of about 400 mg once about every six weeks.

In some aspects, (i) the anti-IL-27 antibody is administered at a dose of about 5 mg/kg once about every three weeks; and (ii) pembrolizumab is administered at a flat dose of about 200 mg once about every two weeks. In some aspects, (i) the anti-IL-27 antibody is administered at a dose of about 5 mg/kg once about every three weeks; and (ii) pembrolizumab is administered at a flat dose of about 200 mg once about every three weeks. In some aspects, (i) the anti-IL-27 antibody is administered at a dose of about 5 mg/kg once about every three weeks; and (ii) pembrolizumab is administered at a flat dose of about 200-400 mg once about every three weeks. In some aspects, (i) the anti-IL-27 antibody is administered at a dose of about 5 mg/kg once about every three weeks; and (ii) pembrolizumab is administered at a flat dose of about 200-400 mg once about every four weeks. In some aspects, (i) the anti-IL-27 antibody is administered at a dose of about 5 mg/kg once about every three weeks; and (ii) pembrolizumab is administered at a flat dose of about 200-400 mg once about every six weeks. In some aspects, (i) the anti-IL-27 antibody is administered at a dose of about 5 mg/kg once about every three weeks; and (ii) pembrolizumab is administered at a flat dose of about 400 mg/kg once about every four weeks. In some aspects, (i) the anti-IL-27 antibody is administered at a dose of about 5 mg/kg once about every three weeks; and (ii) pembrolizumab is administered at a flat dose of about 400 mg once about every six weeks.

In some aspects, (i) the anti-IL-27 antibody is administered at a dose of about 6 mg/kg once about every three weeks; and (ii) pembrolizumab is administered at a flat dose of about 200 mg once about every two weeks. In some aspects, (i) the anti-IL-27 antibody is administered at a dose of about 6 mg/kg once about every three weeks; and (ii) pembrolizumab is administered at a flat dose of about 200 mg once about every three weeks. In some aspects, (i) the anti-IL-27 antibody is administered at a dose of about 6 mg/kg once about every three weeks; and (ii) pembrolizumab is administered at a flat dose of about 200-400 mg once about every three weeks. In some aspects, (i) the anti-IL-27 antibody is administered at a dose of about 6 mg/kg once about every three weeks; and (ii) pembrolizumab is administered at a flat dose of about 200-400 mg once about every four weeks. In some aspects, (i) the anti-IL-27 antibody is administered at a dose of about 6 mg/kg once about every three weeks; and (ii) pembrolizumab is administered at a flat dose of about 200-400 mg once about every six weeks. In some aspects, (i) the anti-IL-27 antibody is administered at a dose of about 6 mg/kg once about every three weeks; and (ii) pembrolizumab is administered at a flat dose of about 400 mg once about every four weeks. In some aspects, (i) the anti-IL-27 antibody is administered at a dose of about 6 mg/kg once about every three weeks; and (ii) pembrolizumab is administered at a flat dose of about 400 mg once about every six weeks.

In some aspects, (i) the anti-IL-27 antibody is administered at a dose of about 7 mg/kg once about every three weeks; and (ii) pembrolizumab is administered at a flat dose of about 200 mg once about every two weeks. In some aspects, (i) the anti-IL-27 antibody is administered at a dose of about 7 mg/kg once about every three weeks; and (ii) pembrolizumab is administered at a flat dose of about 200 mg once about every three weeks. In some aspects, (i) the anti-IL-27 antibody is administered at a dose of about 7 mg/kg once about every three weeks; and (ii) pembrolizumab is administered at a flat dose of about 200-400 mg once about every three weeks. In some aspects, (i) the anti-IL-27 antibody is administered at a dose of about 7 mg/kg once about every three weeks; and (ii) pembrolizumab is administered at a flat dose of about 200-400 mg once about every four weeks. In some aspects, (i) the anti-IL-27 antibody is administered at a dose of about 7 mg/kg once about every three weeks; and (ii) pembrolizumab is administered at a flat dose of about 200-400 mg once about every six weeks. In some aspects, (i) the anti-IL-27 antibody is administered at a dose of about 7 mg/kg once about every three weeks; and (ii) pembrolizumab is administered at a flat dose of about 400 mg/kg once about every four weeks. In some aspects, (i) the anti-IL-27 antibody is administered at a dose of about 7 mg/kg once about every three weeks; and (ii) pembrolizumab is administered at a flat dose of about 400 mg once about every six weeks.

In some aspects, (i) the anti-IL-27 antibody is administered at a dose of about 8 mg/kg once about every three weeks; and (ii) pembrolizumab is administered at a flat dose of about 200 mg once about every two weeks. In some aspects, (i) the anti-IL-27 antibody is administered at a dose of about 8 mg/kg once about every three weeks; and (ii) pembrolizumab is administered at a flat dose of about 200 mg once about every three weeks. In some aspects, (i) the anti-IL-27 antibody is administered at a dose of about 8 mg/kg once about every three weeks; and (ii) pembrolizumab is administered at a flat dose of about 200-400 mg once about every three weeks. In some aspects, (i) the anti-IL-27 antibody is administered at a dose of about 8 mg/kg once about every three weeks; and (ii) pembrolizumab is administered at a flat dose of about 200-400 mg once about every four weeks. In some aspects, (i) the anti-IL-27 antibody is administered at a dose of about 8 mg/kg once about every three weeks; and (ii) pembrolizumab is administered at a flat dose of about 200-400 mg once about every six weeks. In some aspects, (i) the anti-IL-27 antibody is administered at a dose of about 8 mg/kg once about every three weeks; and (ii) pembrolizumab is administered at a flat dose of about 400 mg once about every four weeks. In some aspects, (i) the anti-IL-27 antibody is administered at a dose of about 8 mg/kg once about every three weeks; and (ii) pembrolizumab is administered at a flat dose of about 400 mg once about every six weeks.

In some aspects, (i) the anti-IL-27 antibody is administered at a dose of about 9 mg/kg once about every three weeks; and (ii) pembrolizumab is administered at a flat dose of about 200 mg once about every two weeks. In some aspects, (i) the anti-IL-27 antibody is administered at a dose of about 9 mg/kg once about every three weeks; and (ii) pembrolizumab is administered at a flat dose of about 200 mg once about every three weeks. In some aspects, (i) the anti-IL-27 antibody is administered at a dose of about 9 mg/kg once about every three weeks; and (ii) pembrolizumab is administered at a flat dose of about 200-400 mg once about every three weeks. In some aspects, (i) the anti-IL-27 antibody is administered at a dose of about 9 mg/kg once about every three weeks; and (ii) pembrolizumab is administered at a flat dose of about 200-400 mg once about every four weeks. In some aspects, (i) the anti-IL-27 antibody is administered at a dose of about 9 mg/kg once about every three weeks; and (ii) pembrolizumab is administered at a flat dose of about 200-400 mg once about every six weeks. In some aspects, (i) the anti-IL-27 antibody is administered at a dose of about 9 mg/kg once about every three weeks; and (ii) pembrolizumab is administered at a flat dose of about 400 mg/kg once about every four weeks. In some aspects, (i) the anti-IL-27 antibody is administered at a dose of about 9 mg/kg once about every three weeks; and (ii) pembrolizumab is administered at a flat dose of about 400 mg once about every six weeks.

In some aspects, (i) the anti-IL-27 antibody is administered at a dose of about 10 mg/kg once about every three weeks; and (ii) pembrolizumab is administered at a flat dose of about 200 mg once about every two weeks. In some aspects, (i) the anti-IL-27 antibody is administered at a dose of about 10 mg/kg once about every three weeks; and (ii) pembrolizumab is administered at a flat dose of about 200 mg once about every three weeks. In some aspects, (i) the anti-IL-27 antibody is administered at a dose of about 10 mg/kg once about every three weeks; and (ii) pembrolizumab is administered at a flat dose of about 200-400 mg once about every three weeks. In some aspects, (i) the anti-IL-27 antibody is administered at a dose of about 10 mg/kg once about every three weeks; and (ii) pembrolizumab is administered at a flat dose of about 200-400 mg once about every four weeks. In some aspects, (i) the anti-IL-27 antibody is administered at a dose of about 10 mg/kg once about every three weeks; and (ii) pembrolizumab is administered at a flat dose of about 200-400 mg once about every six weeks. In some aspects, (i) the anti-IL-27 antibody is administered at a dose of about 10 mg/kg once about every three weeks; and (ii) pembrolizumab is administered at a flat dose of about 400 mg once about every four weeks. In some aspects, (i) the anti-IL-27 antibody is administered at a dose of about 10 mg/kg once about every three weeks; and (ii) pembrolizumab is administered at a flat dose of about 400 mg once about every six weeks.

In some aspects, (i) the anti-IL-27 antibody is administered at a dose of about 11 mg/kg once about every three weeks; and (ii) pembrolizumab is administered at a flat dose of about 200 mg once about every two weeks. In some aspects, (i) the anti-IL-27 antibody is administered at a dose of about 11 mg/kg once about every three weeks; and (ii) pembrolizumab is administered at a flat dose of about 200 mg once about every three weeks. In some aspects, (i) the anti-IL-27 antibody is administered at a dose of about 11 mg/kg once about every three weeks; and (ii) pembrolizumab is administered at a flat dose of about 200-400 mg once about every three weeks. In some aspects, (i) the anti-IL-27 antibody is administered at a dose of about 11 mg/kg once about every three weeks; and (ii) pembrolizumab is administered at a flat dose of about 200-400 mg once about every four weeks. In some aspects, (i) the anti-IL-27 antibody is administered at a dose of about 11 mg/kg once about every three weeks; and (ii) pembrolizumab is administered at a flat dose of about 200-400 mg once about every six weeks. In some aspects, (i) the anti-IL-27 antibody is administered at a dose of about 11 mg/kg once about every three weeks; and (ii) pembrolizumab is administered at a flat dose of about 400 mg/kg once about every four weeks. In some aspects, (i) the anti-IL-27 antibody is administered at a dose of about 11 mg/kg once about every three weeks; and (ii) pembrolizumab is administered at a flat dose of about 400 mg once about every six weeks.

In some aspects, (i) the anti-IL-27 antibody is administered at a dose of about 12 mg/kg once about every three weeks; and (ii) pembrolizumab is administered at a flat dose of about 200 mg once about every two weeks. In some aspects, (i) the anti-IL-27 antibody is administered at a dose of about 12 mg/kg once about every three weeks; and (ii) pembrolizumab is administered at a flat dose of about 200 mg once about every three weeks. In some aspects, (i) the anti-IL-27 antibody is administered at a dose of about 12 mg/kg once about every three weeks; and (ii) pembrolizumab is administered at a flat dose of about 200-400 mg once about every three weeks. In some aspects, (i) the anti-IL-27 antibody is administered at a dose of about 12 mg/kg once about every three weeks; and (ii) pembrolizumab is administered at a flat dose of about 200-400 mg once about every four weeks. In some aspects, (i) the anti-IL-27 antibody is administered at a dose of about 12 mg/kg once about every three weeks; and (ii) pembrolizumab is administered at a flat dose of about 200-400 mg once about every six weeks. In some aspects, (i) the anti-IL-27 antibody is administered at a dose of about 12 mg/kg once about every three weeks; and (ii) pembrolizumab is administered at a flat dose of about 400 mg once about every four weeks. In some aspects, (i) the anti-IL-27 antibody is administered at a dose of about 12 mg/kg once about every three weeks; and (ii) pembrolizumab is administered at a flat dose of about 400 mg once about every six weeks.

In some aspects, (i) the anti-IL-27 antibody is administered at a dose of about 13 mg/kg once about every three weeks; and (ii) pembrolizumab is administered at a flat dose of about 200 mg once about every two weeks. In some aspects, (i) the anti-IL-27 antibody is administered at a dose of about 13 mg/kg once about every three weeks; and (ii) pembrolizumab is administered at a flat dose of about 200 mg once about every three weeks. In some aspects, (i) the anti-IL-27 antibody is administered at a dose of about 13 mg/kg once about every three weeks; and (ii) pembrolizumab is administered at a flat dose of about 200-400 mg once about every three weeks. In some aspects, (i) the anti-IL-27 antibody is administered at a dose of about 13 mg/kg once about every three weeks; and (ii) pembrolizumab is administered at a flat dose of about 200-400 mg once about every four weeks. In some aspects, (i) the anti-IL-27 antibody is administered at a dose of about 13 mg/kg once about every three weeks; and (ii) pembrolizumab is administered at a flat dose of about 200-400 mg once about every six weeks. In some aspects, (i) the anti-IL-27 antibody is administered at a dose of about 13 mg/kg once about every three weeks; and (ii) pembrolizumab is administered at a flat dose of about 400 mg/kg once about every four weeks. In some aspects, (i) the anti-IL-27 antibody is administered at a dose of about 13 mg/kg once about every three weeks; and (ii) pembrolizumab is administered at a flat dose of about 400 mg once about every six weeks.

In some aspects, (i) the anti-IL-27 antibody is administered at a dose of about 14 mg/kg once about every three weeks; and (ii) pembrolizumab is administered at a flat dose of about 200 mg once about every two weeks. In some aspects, (i) the anti-IL-27 antibody is administered at a dose of about 14 mg/kg once about every three weeks; and (ii) pembrolizumab is administered at a flat dose of about 200 mg once about every three weeks. In some aspects, (i) the anti-IL-27 antibody is administered at a dose of about 14 mg/kg once about every three weeks; and (ii) pembrolizumab is administered at a flat dose of about 200-400 mg once about every three weeks. In some aspects, (i) the anti-IL-27 antibody is administered at a dose of about 14 mg/kg once about every three weeks; and (ii) pembrolizumab is administered at a flat dose of about 200-400 mg once about every four weeks. In some aspects, (i) the anti-IL-27 antibody is administered at a dose of about 14 mg/kg once about every three weeks; and (ii) pembrolizumab is administered at a flat dose of about 200-400 mg once about every six weeks. In some aspects, (i) the anti-IL-27 antibody is administered at a dose of about 14 mg/kg once about every three weeks; and (ii) pembrolizumab is administered at a flat dose of about 400 mg once about every four weeks. In some aspects, (i) the anti-IL-27 antibody is administered at a dose of about 14 mg/kg once about every three weeks; and (ii) pembrolizumab is administered at a flat dose of about 400 mg once about every six weeks.

In some aspects, (i) the anti-IL-27 antibody is administered at a dose of about 15 mg/kg once about every three weeks; and (ii) pembrolizumab is administered at a flat dose of about 200 mg once about every two weeks. In some aspects, (i) the anti-IL-27 antibody is administered at a dose of about 15 mg/kg once about every three weeks; and (ii) pembrolizumab is administered at a flat dose of about 200 mg once about every three weeks. In some aspects, (i) the anti-IL-27 antibody is administered at a dose of about 15 mg/kg once about every three weeks; and (ii) pembrolizumab is administered at a flat dose of about 200-400 mg once about every three weeks. In some aspects, (i) the anti-IL-27 antibody is administered at a dose of about 15 mg/kg once about every three weeks; and (ii) pembrolizumab is administered at a flat dose of about 200-400 mg once about every four weeks. In some aspects, (i) the anti-IL-27 antibody is administered at a dose of about 15 mg/kg once about every three weeks; and (ii) pembrolizumab is administered at a flat dose of about 200-400 mg once about every six weeks. In some aspects, (i) the anti-IL-27 antibody is administered at a dose of about 15 mg/kg once about every three weeks; and (ii) pembrolizumab is administered at a flat dose of about 400 mg/kg once about every four weeks. In some aspects, (i) the anti-IL-27 antibody is administered at a dose of about 15 mg/kg once about every three weeks; and (ii) pembrolizumab is administered at a flat dose of about 400 mg once about every six weeks.

In some aspects, (i) the anti-IL-27 antibody is administered at a dose of about 16 mg/kg once about every three weeks; and (ii) pembrolizumab is administered at a flat dose of about 200 mg once about every two weeks. In some aspects, (i) the anti-IL-27 antibody is administered at a dose of about 16 mg/kg once about every three weeks; and (ii) pembrolizumab is administered at a flat dose of about 200 mg once about every three weeks. In some aspects, (i) the anti-IL-27 antibody is administered at a dose of about 16 mg/kg once about every three weeks; and (ii) pembrolizumab is administered at a flat dose of about 200-400 mg once about every three weeks. In some aspects, (i) the anti-IL-27 antibody is administered at a dose of about 16 mg/kg once about every three weeks; and (ii) pembrolizumab is administered at a flat dose of about 200-400 mg once about every four weeks. In some aspects, (i) the anti-IL-27 antibody is administered at a dose of about 16 mg/kg once about every three weeks; and (ii) pembrolizumab is administered at a flat dose of about 200-400 mg once about every six weeks. In some aspects, (i) the anti-IL-27 antibody is administered at a dose of about 16 mg/kg once about every three weeks; and (ii) pembrolizumab is administered at a flat dose of about 400 mg once about every four weeks. In some aspects, (i) the anti-IL-27 antibody is administered at a dose of about 16 mg/kg once about every three weeks; and (ii) pembrolizumab is administered at a flat dose of about 400 mg once about every six weeks.

In some aspects, (i) the anti-IL-27 antibody is administered at a dose of about 17 mg/kg once about every three weeks; and (ii) pembrolizumab is administered at a flat dose of about 200 mg once about every two weeks. In some aspects, (i) the anti-IL-27 antibody is administered at a dose of about 17 mg/kg once about every three weeks; and (ii) pembrolizumab is administered at a flat dose of about 200 mg once about every three weeks. In some aspects, (i) the anti-IL-27 antibody is administered at a dose of about 17 mg/kg once about every three weeks; and (ii) pembrolizumab is administered at a flat dose of about 200-400 mg once about every three weeks. In some aspects, (i) the anti-IL-27 antibody is administered at a dose of about 17 mg/kg once about every three weeks; and (ii) pembrolizumab is administered at a flat dose of about 200-400 mg once about every four weeks. In some aspects, (i) the anti-IL-27 antibody is administered at a dose of about 17 mg/kg once about every three weeks; and (ii) pembrolizumab is administered at a flat dose of about 200-400 mg once about every six weeks. In some aspects, (i) the anti-IL-27 antibody is administered at a dose of about 17 mg/kg once about every three weeks; and (ii) pembrolizumab is administered at a flat dose of about 400 mg/kg once about every four weeks. In some aspects, (i) the anti-IL-27 antibody is administered at a dose of about 17 mg/kg once about every three weeks; and (ii) pembrolizumab is administered at a flat dose of about 400 mg once about every six weeks.

In some aspects, (i) the anti-IL-27 antibody is administered at a dose of about 18 mg/kg once about every three weeks; and (ii) pembrolizumab is administered at a flat dose of about 200 mg once about every two weeks. In some aspects, (i) the anti-IL-27 antibody is administered at a dose of about 18 mg/kg once about every three weeks; and (ii) pembrolizumab is administered at a flat dose of about 200 mg once about every three weeks. In some aspects, (i) the anti-IL-27 antibody is administered at a dose of about 18 mg/kg once about every three weeks; and (ii) pembrolizumab is administered at a flat dose of about 200-400 mg once about every three weeks. In some aspects, (i) the anti-IL-27 antibody is administered at a dose of about 18 mg/kg once about every three weeks; and (ii) pembrolizumab is administered at a flat dose of about 200-400 mg once about every four weeks. In some aspects, (i) the anti-IL-27 antibody is administered at a dose of about 18 mg/kg once about every three weeks; and (ii) pembrolizumab is administered at a flat dose of about 200-400 mg once about every six weeks. In some aspects, (i) the anti-IL-27 antibody is administered at a dose of about 18 mg/kg once about every three weeks; and (ii) pembrolizumab is administered at a flat dose of about 400 mg once about every four weeks. In some aspects, (i) the anti-IL-27 antibody is administered at a dose of about 18 mg/kg once about every three weeks; and (ii) pembrolizumab is administered at a flat dose of about 400 mg once about every six weeks.

In some aspects, (i) the anti-IL-27 antibody is administered at a dose of about 19 mg/kg once about every three weeks; and (ii) pembrolizumab is administered at a flat dose of about 200 mg once about every two weeks. In some aspects, (i) the anti-IL-27 antibody is administered at a dose of about 19 mg/kg once about every three weeks; and (ii) pembrolizumab is administered at a flat dose of about 200 mg once about every three weeks. In some aspects, (i) the anti-IL-27 antibody is administered at a dose of about 19 mg/kg once about every three weeks; and (ii) pembrolizumab is administered at a flat dose of about 200-400 mg once about every three weeks. In some aspects, (i) the anti-IL-27 antibody is administered at a dose of about 19 mg/kg once about every three weeks; and (ii) pembrolizumab is administered at a flat dose of about 200-400 mg once about every four weeks. In some aspects, (i) the anti-IL-27 antibody is administered at a dose of about 19 mg/kg once about every three weeks; and (ii) pembrolizumab is administered at a flat dose of about 200-400 mg once about every six weeks. In some aspects, (i) the anti-IL-27 antibody is administered at a dose of about 19 mg/kg once about every three weeks; and (ii) pembrolizumab is administered at a flat dose of about 400 mg/kg once about every four weeks. In some aspects, (i) the anti-IL-27 antibody is administered at a dose of about 19 mg/kg once about every three weeks; and (ii) pembrolizumab is administered at a flat dose of about 400 mg once about every six weeks.

In some aspects, (i) the anti-IL-27 antibody is administered at a dose of about 20 mg/kg once about every three weeks; and (ii) pembrolizumab is administered at a flat dose of about 200 mg once about every two weeks. In some aspects, (i) the anti-IL-27 antibody is administered at a dose of about 20 mg/kg once about every three weeks; and (ii) pembrolizumab is administered at a flat dose of about 200 mg once about every three weeks. In some aspects, (i) the anti-IL-27 antibody is administered at a dose of about 20 mg/kg once about every three weeks; and (ii) pembrolizumab is administered at a flat dose of about 200-400 mg once about every three weeks. In some aspects, (i) the anti-IL-27 antibody is administered at a dose of about 20 mg/kg once about every three weeks; and (ii) pembrolizumab is administered at a flat dose of about 200-400 mg once about every four weeks. In some aspects, (i) the anti-IL-27 antibody is administered at a dose of about 20 mg/kg once about every three weeks; and (ii) pembrolizumab is administered at a flat dose of about 200-400 mg once about every six weeks. In some aspects, (i) the anti-IL-27 antibody is administered at a dose of about 20 mg/kg once about every three weeks; and (ii) pembrolizumab is administered at a flat dose of about 400 mg once about every four weeks. In some aspects, (i) the anti-IL-27 antibody is administered at a dose of about 20 mg/kg once about every three weeks; and (ii) pembrolizumab is administered at a flat dose of about 400 mg once about every six weeks.

In some aspects, the anti-IL-27 antibody and pembrolizumab are administered on the same day. In some aspects, the anti-IL-27 antibody and pembrolizumab are administered concurrently. In some aspects, the anti-IL-27 antibody and pembrolizumab are administered sequentially. In some aspects, the anti-IL-27 antibody is administered before pembrolizumab. In some aspects, the anti-IL-27 antibody is administered after pembrolizumab. In some aspects, the anti-IL-27 antibody and pembrolizumab are administered on different days.

### C. Pharmaceutical Compositions and Formulations

In some aspects, the antibody, or antigen-binding portion thereof, useful in the methods and compositions disclosed herein is present in a pharmaceutical composition. As such, some aspects of the present disclosure are directed to a pharmaceutical composition comprising an anti-IL-27 antibody with a pharmaceutically acceptable diluent, carrier, solubilizer, emulsifier, preservative and/or adjuvant. In some aspects, the anti-IL-27 antibody and pembrolizumab are present in separate pharmaceutical compositions. In some aspects, the anti-IL-27 antibody and pembrolizumab are present in a single formulation, i.e., a co-formulation of the anti-IL-27 antibody and pembrolizumab.

In certain aspects, acceptable formulation materials preferably are nontoxic to recipients at the dosages and concentrations employed.

In certain aspects, the formulation material(s) are for s.c. and/or I.V. administration. In certain aspects, the primary vehicle or carrier in a pharmaceutical composition is either aqueous or non-aqueous in nature. For example, in certain aspects, a suitable vehicle or carrier is water for injection, physiological saline solution or artificial cerebrospinal fluid, possibly supplemented with other materials common in compositions for parenteral administration. In certain aspects, the saline comprises isotonic phosphate-buffered saline. In certain aspects, neutral buffered saline or saline mixed with serum albumin are further exemplary vehicles. In certain aspects, pharmaceutical compositions comprise Tris buffer of about pH 7.0-8.5, or acetate buffer of about pH 4.0-5.5. In some aspects, the pharmaceutical composition further comprises sorbitol or a suitable substitute therefore. In certain aspects, a composition comprising an anti-IL-27 antibody is prepared for storage by mixing the selected composition having the desired degree of purity with optional formulation agents (Remington's Pharmaceutical Sciences, supra) in the form of a lyophilized cake or an aqueous solution. Further, in certain aspects, a composition comprising an anti-IL-27 antibody is formulated as a lyophilizate using appropriate excipients such as sucrose.

In certain aspects, the pharmaceutical composition is selected for parenteral delivery. In certain aspects, the compositions is selected for inhalation or for delivery through the digestive tract, such as orally. The preparation of such pharmaceutically acceptable compositions is within the ability of one skilled in the art.

In certain aspects, the formulation components are present in concentrations that are acceptable to the site of administration. In certain aspects, buffers are used to maintain the composition at physiological pH or at a slightly lower pH, typically within a pH range of from about 5 to about 8.

In certain aspects, when parenteral administration is contemplated, a therapeutic composition is in the form of a pyrogen-free, parenterally acceptable aqueous solution comprising an anti-IL-27 antibody, in a pharmaceutically acceptable vehicle. In certain aspects, a vehicle for parenteral injection is sterile distilled water in which an anti-IL-27 antibody is formulated as a sterile, isotonic solution, and properly preserved. In certain aspects, the preparation involves the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain aspects, hyaluronic acid is also used. Hyaluronic acid, when present, can have the effect of promoting sustained duration in the circulation. In certain aspects, implantable drug delivery devices are used to introduce the desired molecule.

In certain aspects, a pharmaceutical composition is formulated for inhalation. In certain aspects, an anti-IL-27 antibody is formulated as a dry powder for inhalation. In certain aspects, an inhalation solution comprising an anti-IL-27 antibody is formulated with a propellant for aerosol delivery. In certain aspects, solutions are nebulized. Pulmonary administration is further described in PCT application No. PCT/US94/001875, which describes pulmonary delivery of chemically modified proteins.

In certain aspects, the pharmaceutical composition disclosed herein is formulated for oral administration. In some aspects, the pharmaceutical composition is administered orally. In certain aspects, an anti-IL-27 antibody that is administered in this fashion is formulated with or without those carriers customarily used in the compounding of solid dosage forms such as tablets and capsules. In certain aspects, a capsule is designed to release the active portion of the formulation at the point in the gastrointestinal tract when bioavailability is maximized and pre-systemic degradation is minimized. In certain aspects, at least one additional agent is included to facilitate absorption of an anti-IL-27 antibody. In certain aspects, diluents, flavorings, low melting point waxes, vegetable oils, lubricants, suspending agents, tablet disintegrating agents, and binders are also employed.

In certain aspects, a pharmaceutical composition involves an effective quantity of an anti-IL-27 antibody in a mixture with non-toxic excipients which are suitable for the manufacture of tablets. In certain aspects, by dissolving the tablets in sterile water, or another appropriate vehicle, solutions are prepared in unit-dose form. In certain aspects, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an anti-IL-27 antibody in sustained- or controlled-delivery formulations. In certain aspects, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain aspects, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain aspects, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain aspects, this is accomplished by filtration through sterile filtration membranes. In certain aspects, where the composition is lyophilized, sterilization using this method is conducted either prior to or following lyophilization and reconstitution. In certain aspects, the composition for parenteral administration is stored in lyophilized form or in a solution. In certain aspects, parenteral compositions generally are placed into a container having a sterile access port, for example, an intravenous solution bag or vial having a stopper pierceable by a hypodermic injection needle.

In certain aspects, once the pharmaceutical composition has been formulated, it is stored in sterile vials as a solution, suspension, gel, emulsion, solid, or as a dehydrated or lyophilized powder. In certain aspects, such formulations are stored either in a ready-to-use form or in a form (e.g., lyophilized) that is reconstituted prior to administration.

In certain aspects, kits are provided for producing a single-dose administration unit. In certain aspects, the kit comprises both a first container having a dried protein and a second container having an aqueous formulation. In certain aspects, kits comprising single and multi-chambered pre-filled syringes (e.g., liquid syringes and lyosyringes) are included.

### EXAMPLES

While the present disclosure has been described with reference to the specific aspects thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1: In Vivo Administration of Anti-IL-27 Antibody in Combination with Pembrolizumab in Patients with Advanced Solid Tumors

A dose-escalation study (accelerated single patient followed by standard 3+3) enrolled patients (pts) with advanced treatment-refractory solid tumors to test the in vivo effects of administering a fully human IgG1 blocking antibody to IL-27 (SRF388) alone or in combination with pembrolizumab. Patients with treatment-refractory non-small cell lung cancer (NSCLC), clear cell renal cell carcinoma (ccRCC), and hepatocellular carcinoma (HCC) were enrolled. Based on safety, tolerability, pharmacokinetics (PK), peripheral pSTAT1 inhibition, and preliminary efficacy, the dosing regimen for SRF388 monotherapy was selected as 10 mg/mL every 4 weeks. The dosing regimen for the combination therapy was selected as 10 mg/kg of SRF388 and 200 mg pembrolizumab every 3 weeks.

The SRF388+pembrolizumab combination group enrolled 10 patients. Of the 9 patients evaluated, 3 were diagnosed with ccRCC and 6 were diagnosed with HCC. Median age was 69 years. Most patients were male (90%). Most patients had an ECOG PS of 1 (80%). Approximately 60% had prior PD-(L)1 blockade, and 20% had ≥5 prior therapies. Baseline patient and disease characteristics of the combination group are shown in Table 3.

TABLE 3
Baseline Patient & Disease Characteristics

| Demographics | | SRF388 + Pembro |
|---|---|---|
| n (%) | | (n = 10) |
| Age | Median years | 69 (58, 74) |
| | (range) | |
| Gender | Female | 1 (10.0) |
| | Male | 9 (90.0) |
| Race | Asian | 6 (60.0) |
| | Black or African | 1 (10.0) |
| | American | |
| | White | 3 (30.0) |
| | Unknown | 0 (0.0) |
| | Not Reported | 0 (0.0) |
| ECOG PS | 0 | 2 (20.0) |
| | 1 | 8 (80.0) |
| Median time since initial | | 58 (24, 132) |
| diagnosis, months (range) | | |
| Lines of | 1 | 0 (0.0) |
| Prior Systemic | 2 | 5 (50.0) |
| Therapy | 3-4 | 3 (30.0) |
| | ≥5 | 2 (20.0) |
| Prior | Yes | 6 (60.0) |
| aPD-1/aPD-L1 | No | 4 (40.0) |
| IMDC* | Favorable | 1 (33.3) |
| (ccRCC | Intermediate | 1 (33.3) |
| patients only) | Poor | 0 (0.0) |

*One or more patients are missing data.

The combination therapy was found to be well tolerated. The number of adverse events (AEs) observed in the combination group is shown in Table 4. In this study, a treatment-emergent adverse event (TEAE) is defined as an adverse event that emerges or worsens in the period from the first dose of SRF388 to 30 days after the last dose. No Grade≥3 were TEAEs were observed in any patients in the combination therapy group.

TABLE 4
Adverse events observed in the combination therapy group.

| Adverse | Grade | Grade | Grade | Grade | Grade | All |
|---|---|---|---|---|---|---|
| Event | 1 | 2 | 3 | 4 | 5 | Grades |
| (AE) | (n, %) | (n, %) | (n, %) | (n, %) | (n, %) | (n, %) |
| Decreased | 1 (10.0) | 0 | 0 | 0 | 0 | 1 (10.0) |
| appetite | | | | | | |
| Dyspnoea | 1 (10.0) | 0 | 0 | 0 | 0 | 1 (10.0) |
| Back pain | 1 (10.0) | 1 (10.0) | 0 | 0 | 0 | 2 (20.0) |
| Anaemia | 1 (10.0) | 0 | 0 | 0 | 0 | 1 (10.0) |
| Hypothyroidism | 1 (10.0) | 1 (10.0) | 0 | 0 | 0 | 2 (20.0) |
| Hyperuricaemia | 2 (20.0) | 0 | 0 | 0 | 0 | 2 (20.0) |

Efficacy of the combination therapy was evaluated by measuring tumor response in each patient. Tumor response was assessed by RECIST1.1 (see, e.g., Eisenhaur et al. Eur J Cancer 45 (2009): 228-247). Imaging assessments were performed every 9 weeks.

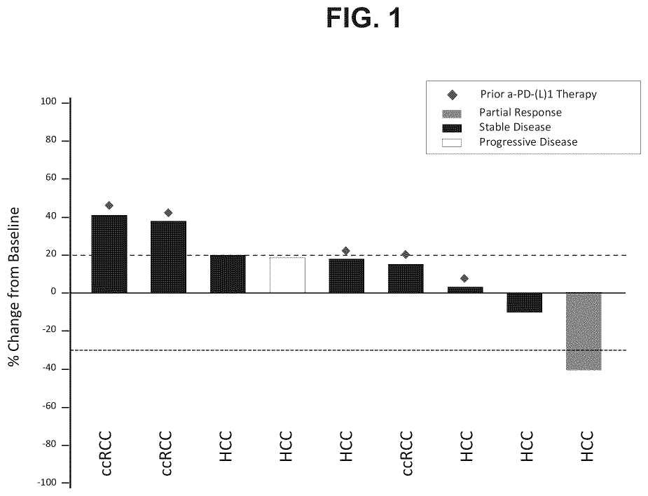

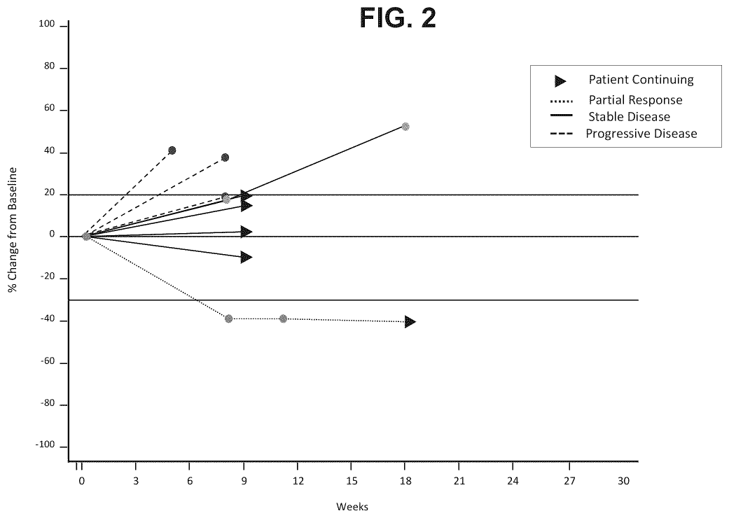

A summary of patient response (n=9) with the combination therapy is shown in FIG. 1 and FIG. 2. Tumor response is presented as best percent change from baseline in sum of target lesions (FIG. 1) and target lesion change over time (FIG. 2). The combination therapy produced a 67% disease control rate in this patient group (5 patients with stable disease and 1 patient with partial response). Notably, one patient with HCC refractory to two prior treatments with vascular endothelial growth factor receptor tyrosine kinase inhibitor (VEGFR-TKI) experienced a confirmed partial response (PR) (see FIG. 1, far right column; FIG. 2, dotted line).

These results demonstrate the therapeutic potential of the combination of IL-27 blockade (SRF388) and PD-1 blockade (pembrolizumab) in treatment refractory NSCLC, ccRCC, and HCC.

1. A method of stimulating an immune response in a subject, the method comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

2. A method of treating a cancer in a subject in need thereof comprising administering to the subject (i) an antibody that binds human IL-27 or an antigen binding portion thereof (“an anti-IL-27 antibody”), and (ii) pembrolizumab; wherein the anti-IL-27 antibody comprises a heavy chain CDR1 comprising the amino acid sequence set forth in SEQ ID NO: 5, a heavy chain CDR2 comprising the amino acid sequence set forth in SEQ ID NO: 6, a heavy chain CDR3 comprising the amino acid sequence set forth in SEQ ID NO: 7, a light chain CDR1 comprising the amino acid sequence set forth in SEQ ID NO: 13, a light chain CDR2 comprising the amino acid sequence set forth in SEQ ID NO: 14, and a light chain CDR3 comprising the amino acid sequence set forth in SEQ ID NO: 15.

3. The method of claim 2, wherein the anti-IL-27 antibody is administered at a dose of at least about 0.003 mg/kg to at least about 20 mg/kg.

4. (canceled)

5. The method of claim 2, wherein the anti-IL-27 antibody is administered once about every week, once about every two weeks, once about every three weeks, once about every four weeks, once about every 6 weeks, once about every 8 weeks, or once about every 12 weeks.

6-18. (canceled)

19. The method of claim 2, wherein pembrolizumab is administered at a dose of at least about 1 mg/kg to at least about 10 mg/kg.

20. (canceled)

21. The method of claim 2, wherein pembrolizumab is administered at a flat dose.

22. (canceled)

23. The method of claim 2, wherein pembrolizumab is administered once about every week, once about every two weeks, once about every three weeks, or once about every four weeks.

24-34. (canceled)

35. The method of claim 2, wherein the anti-IL-27 antibody and pembrolizumab are administered on the same day.

36. The method of claim 2, wherein the anti-IL-27 antibody and pembrolizumab are administered concurrently.

37. The method of claim 2, wherein the anti-IL-27 antibody and pembrolizumab are administered sequentially.

38. The method of claim 2, wherein the anti-IL-27 antibody is administered before pembrolizumab.

39. The method of claim 2, wherein the anti-IL-27 antibody is administered after pembrolizumab.

40. The method of claim 2, wherein the anti-IL-27 antibody and pembrolizumab are administered on different days.

41. The method of claim 2, wherein the anti-IL-27 antibody comprises a heavy chain variable region comprising an amino acid sequence that has at least about 85%, at least about 90%, at least about 95%, at least about 96%, at least about 97%, at least about 98%, [[or]] at least about 99%, or at least 100% sequence identity to the amino acid sequence set forth in SEQ ID NO: 11.

42. (canceled)

43. The method of claim 2, wherein the anti-IL-27 antibody comprises a light chain variable region comprising an amino acid sequence that has at least about 85%, at least about 90%, at least about 95%, at least about 96%, at least about 97%, at least about 98%, at least about 99%, or at least 100% sequence identity to the amino acid sequence set forth in SEQ ID NO: 19.

44. (canceled)

45. (canceled)

46. The method of claim 2, wherein the anti-IL-27 antibody comprises a heavy chain comprising the amino acid sequence set forth in SEQ ID NO: 21.

47. The method of claim 2, wherein the anti-IL-27 antibody comprises a heavy chain comprising the amino acid sequence set forth in SEQ ID NO: 25.

48. The method of claim 2, wherein the anti-IL-27 antibody comprises a light chain comprising the amino acid sequence set forth in SEQ ID NO: 23.

49. (canceled)

50. (canceled)

51. The method of claim 1, wherein the subject is afflicted with a cancer.

52-54. (canceled)

55. The method of claim 2, wherein the subject previously received at least one, at least two, at least three, at least four, or at least five prior anticancer therapies.

56-118. (canceled)


SEQUENCE LISTING (40 sequences)

SEQ ID NO: 1 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 2 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 3 - protein, 636 aa, Homo sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 4 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 5 - protein, 8 aa, synthetic construct
GFTFRSYG

SEQ ID NO: 6 - protein, 8 aa, synthetic construct
ISSSGSYI

SEQ ID NO: 7 - protein, 19 aa, synthetic construct
ARDGGRTSYTATAHNWFDP

SEQ ID NO: 8 - protein, 9 aa, synthetic construct
FTFRSYGMN

SEQ ID NO: 9 - protein, 17 aa, synthetic construct
GISSSGSYIYYADSVKG

SEQ ID NO: 10 - protein, 19 aa, synthetic construct
ARDGGRTSYTATAHNWFDP

SEQ ID NO: 11 - protein, 126 aa, synthetic construct
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSS

SEQ ID NO: 12 - DNA, 378 bp, synthetic construct
  source 1..378: mol_type = other DNA
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 13 - protein, 12 aa, synthetic construct
QSVLFSSNNKNY

SEQ ID NO: 14 - skipped

SEQ ID NO: 15 - protein, 9 aa, synthetic construct
QQHASAPPT

SEQ ID NO: 16 - protein, 17 aa, synthetic construct
KSSQSVLFSSNNKNYLA

SEQ ID NO: 17 - protein, 7 aa, synthetic construct
WASTRES

SEQ ID NO: 18 - protein, 9 aa, synthetic construct
QQHASAPPT

SEQ ID NO: 19 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHASAPPTFGGGTKVEIK

SEQ ID NO: 20 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttattcagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagcacgccagtgcccctcctacttttggcggagggaccaaggttgagatcaaa

SEQ ID NO: 21 - protein, 456 aa, synthetic construct
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 1368 bp, synthetic construct
  source 1..1368: mol_type = other DNA
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 23 - protein, 220 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHASAPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 660 bp, synthetic construct
  source 1..660: mol_type = other DNA
gacatcgtgatgacccagtctccagactccctggctgtgtctctgggcgagagggccaccatcaactgcaagtccagccagagtgttttattcagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagcacgccagtgcccctcctacttttggcggagggaccaaggttgagatcaaacgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 25 - protein, 452 aa, synthetic construct
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - DNA, 1356 bp, synthetic construct
  source 1..1356: mol_type = other DNA
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 27 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 5 aa, synthetic construct
NYYMY

SEQ ID NO: 32 - protein, 17 aa, synthetic construct
GINPSNGGTNFNEKFKN

SEQ ID NO: 33 - protein, 11 aa, synthetic construct
RDYRFDMGFDY

SEQ ID NO: 34 - protein, 15 aa, synthetic construct
RASKGVSTSGYSYLH

SEQ ID NO: 35 - protein, 7 aa, synthetic construct
LASYLES

SEQ ID NO: 36 - protein, 9 aa, synthetic construct
QHSRDLPLT

SEQ ID NO: 37 - protein, 120 aa, synthetic construct [sequence omitted]

SEQ ID NO: 38 - protein, 111 aa, synthetic construct [sequence omitted]

SEQ ID NO: 39 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC